(19) United States
(12) Patent Application Publication
(10) Pub. No.: US 2025/0018061 A1
(43) Pub. Date: Jan. 16, 2025

(54) GENE THERAPY IN SUCCINIC SEMIALDEHYDE DEHYDROGENASE DEFICIENCY (SSADHD)

(71) Applicant: The Children`s Medical Center Corporation, Boston, MA (US)

(72) Inventors: Alexander Rotenberg, Chestnut Hill, MA (US); Hing Cheong Lee, Chestnut Hill, MA (US); Phillip Lawrence Pearl, Wellesley Hills, MA (US)

(21) Appl. No.: 18/714,654

(22) PCT Filed: Dec. 2, 2022
(86) PCT No.: PCT/US2022/080806

§ 371 Date: May 30, 2024

Related U.S. Application Data
(60) Provisional application No. 63/285,432, filed on Dec. 2, 2021.

(51) Int. Cl. A61K 48/00; A61K 38/44; A61P 3/00; A61P 25/08; C12N 15/86

(52) U.S. Cl. CPC A61K 48/0058; A61K 38/44; A61K 48/0075; A61P 3/00; A61P 25/08; C12N 15/86; C12Y 102/01016; C12N 2750/14143

(57) ABSTRACT

Provided herein are methods and compositions for treating succinic semialdehyde dehydrogenase deficiency (SSADHD) using gene therapy to restore, at least partially, expression and/or activity of succinic semialdehyde dehydrogenase (SSADH).

### CLAIM OF PRIORITY

This application claims the benefit of U.S. Provisional Patent Application No. 63/285,432, filed on Dec. 2, 2021, which is incorporated by reference herein in its entirety.

### FEDERALLY SPONSORED RESEARCH OR DEVELOPMENT

This invention was made with Government support under Grant No. NS121858 awarded by the National Institutes of Health. The Government has certain rights in the invention.

### TECHNICAL FIELD

The subject matter disclosed herein generally relates to methods and compositions for treating succinic semialdehyde dehydrogenase deficiency (SSADHD).

### SEQUENCE LISTING

This application contains a Sequence Listing that has been submitted electronically as an XML file named 37314-0119WO1_SL_ST26.xml. The XML file, created on Dec. 1, 2022, is 14,152 bytes in size. The material in the XML file is hereby incorporated by reference in its entirety.

### BACKGROUND

SSADHD is a rare inborn metabolic disorder caused by the functional impairment of succinic semialdehyde dehydrogenase (SSADH; encoded by the ALDH5A1 gene), an enzyme essential for metabolism of the inhibitory neurotransmitter γ-aminobutyric acid (GABA). In SSADHD, pathologic accumulation of GABA and its metabolite γ-hydroxybutyrate (GHB) results in broad spectrum encephalopathy where symptoms often include developmental delay, autism, ataxia, epilepsy, and a heightened risk of sudden unexpected death in epilepsy (SUDEP).

### SUMMARY

The present disclosure is based, at least in part, on the development of gene therapies that restore, at least partially, SSADH in SSADHD patients.

Accordingly, aspects of the present disclosure provide a method of treating succinic semialdehyde dehydrogenase deficiency (SSADHD) in a subject, the method comprising administering to the subject an effective amount of a polynucleotide comprising a promoter sequence and a sequence encoding succinic semialdehyde dehydrogenase (SSADH).

In some embodiments, the promoter sequence is a neuron-specific promoter sequence. In some embodiments, the promoter sequence is a γ-aminobutyric acid (GABA) transporter 1 (GAT1) promoter sequence, a GABA transporter 3 (GAT1) promoter sequence, a 5-hydroxytryptamin receptor (5HT3R) promoter sequence, a somatostatin (SST) promoter sequence, a parvalbumin (PV) promoter sequence, a Ca²⁻/calmodulin-dependent kinase subunit α (CaMKII) promoter sequence, neuron-specific enolase (NSE) promoter sequence, synapsin I with a minimal CMV sequence (Syn I-minCMV) promoter sequence, or a aldehyde dehydrogenase 5 family member A1 (ALDH5A1) promoter sequence. In some embodiments, the promoter sequence comprises a nucleotide sequence having at least 90%, at least 95%, at least 98%, at least 99% or 100% identity to SEQ ID NO:2.

In some embodiments, the sequence encoding SSADH comprises a nucleotide sequence having at least 90%, at least 95%, at least 98%, at least 99% or 100% identity to SEQ ID NO:1.

In some embodiments, the polynucleotide comprises a nucleotide sequence having at least 90%, at least 95%, at least 98%, at least 99% or 100% identity to SEQ ID NO:3.

In some embodiments, the polynucleotide is administered to the central nervous system (CNS) of the subject. In some embodiments, the administering of the polynucleotide results in expression of SSADH in the brain of the subject. In some embodiments, the administering of the polynucleotide results in expression of SSADH in parvalbumin-positive interneurons of the subject.

In some embodiments, the subject is a human.

In some embodiments, the polynucleotide is a viral vector. In some embodiments, the viral vector is a lentivirus vector, an alphavirus vector, an enterovirus vector, a pestivirus vector, a baculovirus vector, a herpesvirus vector, an Epstein Barr virus vector, a papovavirus vector, a poxvirus vector, a vaccinia virus vector, herpes simplex virus vector, an adenovirus vector, or an adeno-associated virus (AAV) vector.

In some embodiments, the (AAV) vector is packaged in an AAV particle. In some embodiments, the AAV particle comprises capsid proteins derived from AAV9 serotype. In some embodiments, the AAV particle comprises a capsid protein variant derived from AAV9 serotype. In some embodiments, the capsid protein variant derived from AAV9 comprises PHP.B capsid or PHP.eB capsid.

Aspects of the present disclosure provide a polynucleotide comprising a nucleotide sequence having at least 90%, at least 95%, at least 98%, at least 99% or 100% identity to SEQ ID NO:3, wherein the nucleotide sequence encodes for succinic semialdehyde dehydrogenase (SSADH).

Aspects of the present disclosure provide an adeno-associated virus (AAV) vector comprising a polynucleotide comprising a nucleotide sequence having at least 90%, at least 95%, at least 98%, at least 99% or 100% identity to SEQ ID NO:3, wherein the nucleotide sequence encodes for SSADH.

Aspects of the present disclosure provide an adeno-associated virus (AAV) particle comprising a polynucleotide encapsidated in an AAV capsid, wherein the polynucleotide comprises a nucleotide sequence having at least 90%, at least 95%, at least 98%, at least 99% or 100% identity to SEQ ID NO:3, wherein the nucleotide sequence encodes for SSADH.

In some embodiments, the AAV capsid comprises capsid proteins derived from AAV9 serotype. In some embodiments, the AAV capsid comprises a capsid protein variant derived from AAV9. In some embodiments, the capsid protein variant derived from AAV9 comprises PHP.B capsid or PHP.eB capsid.

Aspects of the present disclosure provide an adeno-associated virus (AAV) particle for use in a method of treating succinic semialdehyde dehydrogenase deficiency (SSADHD) in a subject, wherein the AAV particle comprises a polynucleotide encapsidated in an AAV capsid, wherein the polynucleotide comprises a nucleotide sequence having at least 90%, at least 95%, at least 98%, at least 99% or 100% identity to SEQ ID NO:3, and wherein the nucleotide sequence encodes for SSADH.

Aspects of the present disclosure provide a composition comprising an adeno-associated virus (AAV) particle, wherein the AAV particle comprises a polynucleotide encapsidated in an AAV capsid, wherein the polynucleotide comprises a nucleotide sequence having at least 90%, at least 95%, at least 98%, at least 99% or 100% identity to SEQ ID NO: 3, wherein the nucleotide sequence encodes for SSADH.

Aspects of the present disclosure provide a method of treating succinic semialdehyde dehydrogenase deficiency (SSADHD) in a subject, the method comprising administering to the subject an effective amount of an AAV particle described herein.

Aspects of the present disclosure provide a method of treating succinic semialdehyde dehydrogenase deficiency (SSADHD), comprising administering to a subject an effective amount of a composition comprising a vector encoding succinic semialdehyde dehydrogenase (SSADH).

In some embodiments, the vector comprises a promoter. In some embodiments, the promoter is the naturally occurring full-length ALDH5A1 gene promoter.

In some embodiments, the vector comprises a promoter and the ALDH5A1 gene or a fragment thereof.

In some embodiments, the promoter is selected from the promoters of
a) GABA transporter 1 (GAT1),
b) GABA transporter 3 (GAT3),
c) 5-hydroxytryptamin receptor (5HT3R),
d) Somatostatin (SST),
e) parvalbumin (PV), and
f) glial fibrillary acidic protein (GFAP).

In some embodiments, the vector is administered to the central nervous system of the subject. In some embodiments, the vector is administered to the subject as a single dose. In some embodiments, the vector is administered to the subject gradually. In some embodiments, the vector is administered in divided doses. In some embodiments, the divided doses are administered sequentially. In some embodiments, the vector is administered to the subject weekly.

In some embodiments, the method leads to normalization of the GABA neurotransmitter. In some embodiments, the method leads to normalization of GABA receptors (e.g., GABA_{A} receptors, GABA_{B} receptors, or both) and related GABA signaling function in the subject.

In some embodiments, the subject is human.

In some embodiments, the vector is a viral vector. In some embodiments, the vector is an AAV (adeno-associated virus) vector. In some embodiments, the vector comprises nucleic acid sequences from a virus (e.g., nucleic acid sequences of viral capsid). In some embodiments, the viral vector has properties that enable blood-brain barrier penetration. In some embodiments the viral vector has tropism for specific cell types.

In some embodiments, the promoter is a cell-type-specific promoter that restricts the expression of SSADH to specific cell types. In some embodiments, the specific cell types comprise inhibitory interneurons (e.g., parvalbumin-positive inhibitory interneurons), astrocytes, or both.

In some embodiments, the vector comprises the ALDH5A1 gene.

Aspects of the present disclosure provide a method of treating SSADHD in a subject, comprising increasing the expression or activity of SSADH in the subject.

In some embodiments, the method comprises administering to a subject an effective amount of a composition comprising a vector encoding SSADH.

In some embodiments, the vector comprises a promoter.

Aspects of the present disclosure provide a method of treating SSADHD in a subject, comprising normalizing GABA receptor and related GABA signaling function in the subject.

In some embodiments, the method comprises administering to a subject an effective amount of a composition comprising a vector encoding SSADH. In some embodiments, the vector comprises a promoter.

### DESCRIPTION OF THE DRAWINGS

FIG. 1 includes a schematic depiction of the GABA metabolic pathway. Cytosolic glutamate is converted by glutamic acid decarboxylase (GAD) to form GABA, which is subsequently translocated into the mitochondria, where GABA is reversibly converted by GABA transaminase (GABA-T) to succinic semialdehyde (SSA). SSA is converted either by SSA reductase (SSAR) to γ-hydroxybutyric acid (GHB), or by SSA dehydrogenase (SSADH) to succinate, which then enters the Krebs cycle. In the absence of SSADH (also referred to as SSADH deficiency), GABA and GHB are accumulated to pathologic levels.

FIG. 2A includes in situ hybridization (ISH) data of aldh5a1 transcripts in adult (P56) C57B1/6J mouse brain. Credit: Allen Brain Institute online database (mouse.brain-map.org/). Note the brain-wide expression of aldh5a1, and its enhanced expression in the hippocampus and the cerebellum.

FIG. 2B includes single-cell RNAseq data of aldh5a1 in mouse cortex and hippocampus. Credit: The Linnarsson lab (linnarssonlab.org/cortex/). Cell types are classified as interneurons and pyramidal cells (PYR).

FIG. 2C includes single-cell RNAseq data of aldh5a1 in the mouse whole cortex and the hippocampus. Credit: Allen Brain Institute online database (celltypes.brain-map.org/rnaseq/mouse_ctx-hip_10λ).

FIG. 3 includes a schematic depiction showing that use-dependent compensatory GABA_{A} receptor expression can trigger seizures in SSADHD and potential enzyme replacement therapy (ERT) response. Under neurotypical situation, balanced levels of GABA and GABA_{A} receptors result in normal inhibitory tone (left panel). In SSADHD, pathologic accumulation of GABA leads to use-dependent reduction of GABA_{A} receptors (middle panel). Despite a hyper-GABAergic condition, overall inhibitory tone is sufficiently impaired, resulting in seizures in SSADHD patients (middle panel). ERT in SSADHD normalizes (or reduces) GABA levels in a setting of reduced GABA_{A} receptors (right panel). Successful ERT outcomes depends on plastic restoration of functional GABA_{A} receptors and inhibitory tone.

FIG. 4 includes a schematic depiction showing some of the key parameters for clinical readiness of SSADH restoration including (1) rate, (2) timing, and (3) cell-type specificity.

FIG. 5A includes a schematic depiction showing construction of the aldh5a1^{lox-riTA-STOP} mouse. The endogenous aldh5a1 gene is disrupted by CRISPR/Cas9-mediated homology directed repair in its first intron with the insertion of a gene cassette containing a splice acceptor (AG) and the rtTA-STOP sequence flanked by two loxP sites (top panel). At baseline, aldh5a1^{lox-rtTA-STOP} mice are SSADH-null due to the disrupted aldh5a1 gene (middle panel). Instead, rtTA expression is driven by endogenous aldh5a1 promoter activities (to combine with a second mouse, TRE-aldh5a1, for doxycycline-mediated rescue strategy). Upon Cre-recombination, aldh5a1 is reconstituted for re-expression (aldh5a1^{Δ}) (bottom panel).

FIG. 5B includes schematic depictions showing conceptual design of a reversible SSADH mouse model. Breeding aldh5a1^{lox-riTA-STOP} and TRE-aldh5a1 mice allows reversible expression of recombinant aldh5a1 in the presence of doxycycline (Dox) tightly driven by a Tet-responsive element (TRE).

FIG. 6A includes a schematic depiction of an experimental paradigm for studying abrupt SSADH restoration in mice.

FIG. 6B includes a schematic depiction of an experimental paradigm for studying gradual SSADH restoration in mice.

FIG. 6C includes a schematic depiction of an experimental paradigm for studying pre-symptomatic SSADH restoration in mice.

FIG. 6D includes a schematic depiction of an experimental paradigm for studying peri-symptomatic SSADH restoration in mice.

FIG. 7A includes representative confocal micrographs showing the cerebellum and the hippocampus at 7 and 14 days post-injection (d.p.i.) in low magnification (10×), using escalating doses of AAV-PHP.B to mimic gradual (top panels), moderate (middle panels), and rapid (bottom panels) transgene expression across various timespans. High magnification (40×) of individual neurons in selected brain regions (labeled Cerebellum (40×) and Hippocampus (40×). Scale bars: 500 μm (10×), 20 μm (40×). Rate-dependent expression was observed at 7 d.p.i across the three dosing schedules (gradual, moderate, and rapid), but these changes were largely diminished at 14 d.p.i. N=4 (per dosing schedule and post-injection time point) from two independent experiments.

FIG. 7B includes a schematic depiction of AAV-GFP administration via IP injection.

FIG. 7C includes a graph showing quantification of GFP intensities from rate-dependent GFP expression via AAV-PHP.B injected across various timespans.

FIG. 7D includes a graph showing GFP+ cells from rate-dependent GFP expression via AAV-PHP.B injected across various timespans.

FIG. 8 includes representative confocal micrographs of cryopreserved brain sections showing AAV-PHP.B-CAG-GFP transduced cells (top row) in the hippocampus and the cerebellum. Immunostaining was performed using various interneuron cellular markers (middle row). Arrow heads indicate GFP-expressing cells co-immunostained by respective interneuron cellular markers (bottom row). Selected identified GFP+ cells are shown in high magnification in insets. Scale bar: 50 μm. VIP, vasoactive intestinal polypeptide-expressing interneurons; PV, parvalbumin.

FIG. 9A includes data from analysis of cortical expression of SSADH. Western blot analysis of cortical lysates from wild-type (WT), heterozygous mutant (HET) aldh5a1^{WT/STOP} mice, and homozygous mutant (HOM) aldh5a1^{STOP/STOP} mice, at postnatal age of 16 days (top panel). β-actin serves as protein loading control. Quantification of SSADH expression is expressed as % WT, showing HOM mice have virtually no SSADH expression.

FIG. 9B includes a graph showing premature lethality of aldh5a1^{STOP/STOP} mice.

FIG. 9C includes a graph showing reduced body weight of aldh5a1^{STOP/STOP} mice at postnatal age of 16 days.

FIG. 9D includes a graph showing reduced body weight of aldh5a1^{STOP/STOP} mice at postnatal age of 10 days to 26 days.

FIG. 9E includes a graph showing differentially (increased or decreased) expressed or unchanged genes from RNA-seq analysis of WT and aldh5a1^{STOP/STOP} mice.

FIG. 9F includes a graph showing autism-associated genes detected from RNA-seq analysis of WT and aldh5a1^{STOP/STOP} mice.

FIG. 9G includes a graph showing KEGG pathway analysis of differentially expressed genes from RNA-seq analysis of WT and aldh5a1^{STOP/STOP} mice.

FIG. 10A includes an image of the movement of WT and aldh5a1^{STOP/STOP} mice.

FIG. 10B includes a graph showing distance traveled by WT and aldh5a1^{STOP/STOP} mice.

FIG. 10C includes an image of the hind limb and tail angle of aldh5a1^{STOP/STOP} mice (left panel) and a graph showing hind limb and tail angle of WT and aldh5a1^{STOP/STOP} mice (right panel).

FIG. 10D includes a graph showing various behaviors of WT and aldh5a1^{STOP/STOP} mice.

FIG. 11A includes a schematic depiction of TdTomato mouse injected with AAV-Cre and brain tissue section showing brain-wide TdTomato fluorescence induced by Cre-mediated recombination.

FIG. 11B includes brain tissue sections showing that PV cells are targetable via AAV-Cre systemic delivery.

FIG. 12A includes a schematic depiction of an aldh5a1^{lox-STOP} mice injected with AAV-Cre.

FIG. 12B includes a graph showing percent survival of WT, aldh5a1^{lox-STOP} mice, and aldh5a1^{lox-STOP} mice administered AAV-Cre.

FIG. 12C includes a graph showing body weight of WT, aldh5a1^{lox-STOP} mice, and aldh5a1^{lox-STOP} mice administered AAV-Cre.

FIG. 12D includes a graph showing body weight of WT and aldh5a1^{lox-STOP} mice administered AAV-Cre.

FIG. 12E includes an image of a western blot showing SSADH expression in wild-type (WT), heterozygous mutant (HET) aldh5a1^{WT/STOP} mice, and homozygous mutant (HOM) aldh5a1^{lox-STOP} mice with and without AAV-Cre administration. β-actin serves as protein loading control.

FIG. 12F includes a graph showing SSADH expression in wild-type (WT), heterozygous mutant (HET) aldh5a1^{WT/STOP} mice, and homozygous mutant (HOM) aldh5a1^{lox-STOP} mice with and without AAV-Cre administration.

FIG. 12G includes a graph showing differentially (increased or decreased) expressed or unchanged genes from RNA-seq analysis of WT and aldh5a1^{lox-STOP} mice administered AAV-Cre.

FIG. 12H includes a graph showing autism-associated genes detected from RNA-seq analysis of WT and aldh5a1^{lox-STOP} mice administered AAV-Cre.

FIG. 12I includes a graph showing KEGG pathway analysis of differentially expressed genes from RNA-seq analysis of WT and aldh5a1^{lox-STOP} mice administered AAV-Cre.

FIG. 13A includes an image of the movement of WT and aldh5a1^{lox-STOP} mice administered AAV-Cre.

FIG. 13B includes a graph showing distance traveled by WT and aldh5a1^{lox-STOP} mice administered AAV-Cre.

FIG. 13C includes a graph showing hind limb and tail angle of WT and aldh5a1^{lox-STOP} mice administered AAV-Cre.

FIG. 13D includes a graph showing various behaviors of WT and aldh5a1^{lox-STOP} mice administered AAV-Cre.

FIG. 14A includes a schematic depiction of crossing of an aldh5a1^{lox-STOP} mice with a PV-Cre mouse.

FIG. 14B includes a graph showing percent survival of WT, HOM mice, and HOM; PV^{cre+} mice.

FIG. 14C includes a graph showing body weight of WT, HOM; PV^{Cre-/+} mice, and HOM; PV^{Cre+/+} mice.

FIG. 14D includes a graph showing body weight of WT, HOM; PV^{Cre-/+} mice, and HOM; PV^{Cre+/+} mice.

FIG. 14E includes a graph showing differentially (increased or decreased) expressed or unchanged genes from RNA-seq analysis of HOM; PV^{Cre} mice and WT; PV^{Cre} mice.

FIG. 14F includes a graph showing autism-associated genes detected from RNA-seq analysis of HOM; PV^{Cre} mice and WT; PV^{Cre} mice.

FIG. 14G includes a graph showing KEGG pathway analysis of differentially expressed genes from RNA-seq analysis of HOM; PV^{Cre} mice and WT; PV^{Cre} mice.

FIG. 15A includes an image of a western blot showing SSADH expression in WT, HOM; PV^{Cre-/+} mice, HOM; PV^{Cre+/+} mice, and HOM; PV^{Cre+} mice. β-actin serves as protein loading control.

FIG. 15B includes a graph showing SSADH expression in WT, HOM; PV^{Cre-/+} mice, HOM; PV^{Cre+/+} mice, and HOM; PV^{Cre/+} mice.

FIG. 15C includes a graph showing SSADH (% WT) plotted against age of death for HOM; PV^{Cre-/+} mice and HOM; PV^{cre+} mice.

FIG. 16A includes an image of the movement of HET; PV^{Cre-/+} mice and HOM; PV^{Cre-/+} mice.

FIG. 16B includes a graph showing distance traveled by HET; PV^{Cre-/+} mice and HOM; PV^{Cre-/+} mice.

FIG. 16C includes a graph showing hind limb and tail angle of HET; PV^{Cre-/+} mice and HOM; PV^{Cre-/+} mice.

FIG. 16D includes a graph showing various behaviors of HET; PV^{Cre-/+} mice and HOM; PV^{Cre-/+} mice.

FIG. 17A includes electroencephalogram (EEG) recordings showing that brain-wide SSADH restoration suppresses seizures.

FIG. 17B includes images of western blots (top) and graphs (bottom) showing SSADH expression in WT mice, HET mice, HOM mice, and HOM mice administered AAV-Cre.

FIG. 18A includes a schematic diagram showing the ˜1.8 kb genomic region of promoter sequence upstream of the ALDH5A1 transcriptional start site. Standard regulatory elements sequence motif search was performed using the Nsite database. The respective locations of these regulatory sites (including motifs found in reverse complement sequence) are listed.

FIG. 18B includes a schematic diagram showing a cloning strategy for pAAV-FLnP-hALDH5A1. The schematic diagram includes an AAV backbone encompassing essential AAV expression and packaging elements (left). The human ALDH5A1 promoter will be subcloned into the AAV backbone to form the pAAV-FLnP intermediate (middle). The recombinant ALDH5A1 gene (coding sequence only) will be further inserted via restriction enzyme digestion and re-ligation to form the pAAV-FlnP-hALDH5A1 gene therapy vector (right).

### DETAILED DESCRIPTION

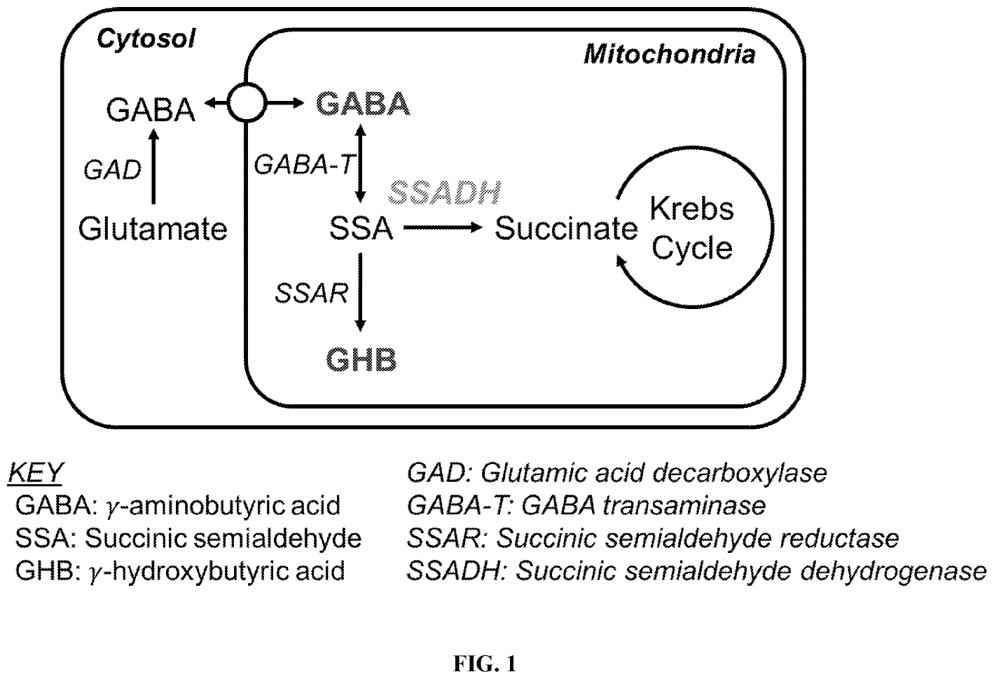

SSADHD is a rare autosomal recessive metabolic disorder (prevalence: ˜200 documented cases worldwide, with most cases concentrated in North America) caused by loss of function mutations in the aldehyde dehydrogenase 5 family member A1 (ALDH5A1) gene. ALDH5A1 encodes SSADH, which is essential for metabolic conversion of the inhibitory neurotransmitter γ-aminobutyric acid (GABA) (FIG. 1). In the absence of SSADH, GABA and its metabolite γ-hydroxybutyrate (GHB) accumulate to pathologic levels in the brain, resulting in non-progressive broad-spectrum encephalopathy. Paradoxically, despite profound increase in extrasynaptic GABA, patients with SSADHD experience frequent seizures and significant risk of sudden unexpected death in epilepsy (SUDEP) in a hyper-GABAergic state. This likely results from use-dependent compensatory downregulation of GABA_{A} and (to a certain extent) GABA_{B} receptors. To date, treatment for SSADHD is symptomatic. A therapy that addresses the underlying enzyme deficiency in SSADHD is absent.

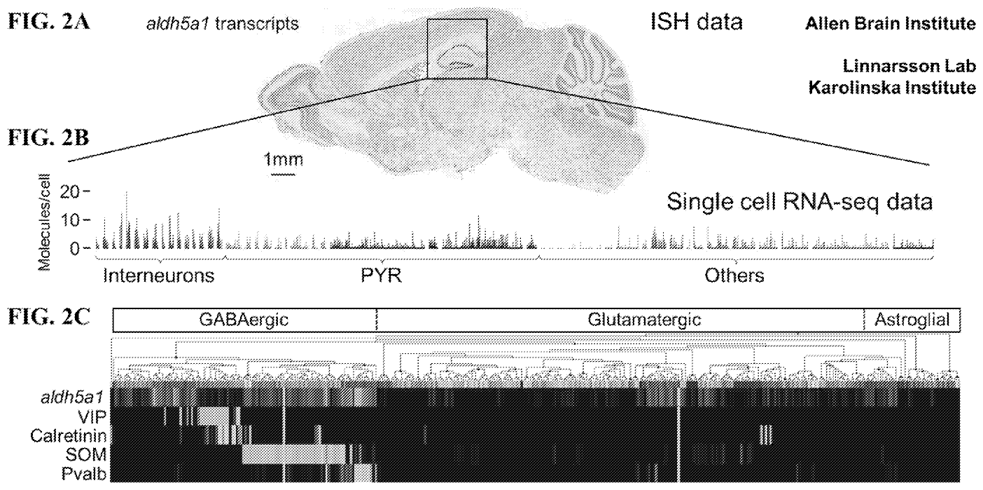

As shown in FIGS. 2A-2C, wild-type SSADH expression appears to be biased towards certain cell populations in the hippocampus and the cerebellum^{31,32}. Therefore, global SSADH restoration might risk adverse effects due to non-specific reduction in GABAergic signaling. If so, then limiting SSADH restoration to relevant brain regions can be safer and sufficient to rescue SSADHD. Experiments described herein tested the safety and efficacy of regional and global SSADH restoration, showing that brain region-directed SSADH restoration is sufficient for SSADHD phenotype reversal.

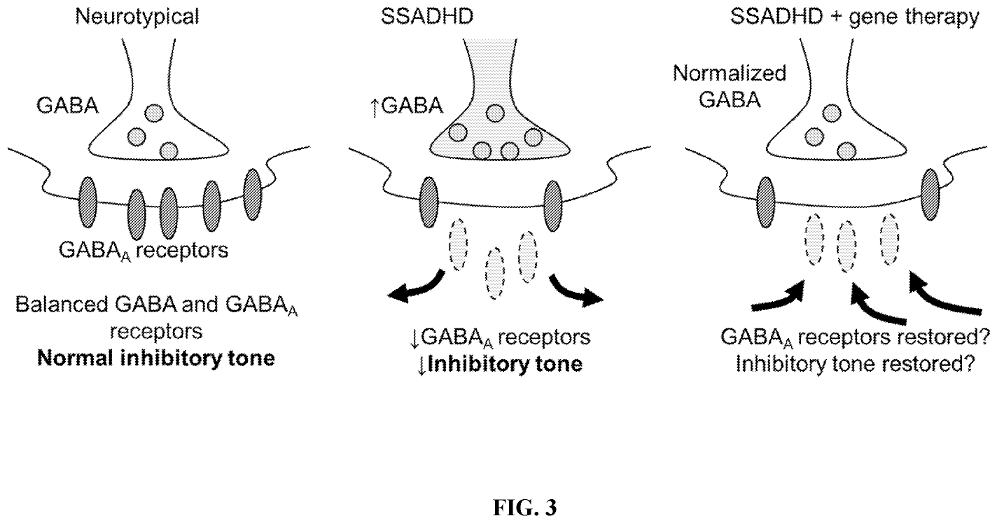

Proof-of-concept experimental enzyme replacement therapy (ERT)¹⁴ or liver-directed adenoviral aldh5a1 gene transfer¹⁵ increases survival of aldh5a1^{-/-} mice. This raises realistic prospects for clinical SSADH-restoring therapies. However, the profound reductions in GABA catabolism and altered signaling in SSADHD fundamentally impact brain development. Brain plasticity and the status of GABAergic functions might play a key role in determining the outcomes of such SSADH-restoring strategies. Postsynaptic GABAergic responses undergo an early developmental switch from excitation to inhibition mediated by tight regulation of chloride homeostasis¹⁶⁻¹⁸ In SSADHD, altered chloride homeostasis might lead to depolarizing GABAergic neurotransmission¹⁴. It is not known how SSADH restoration might impact neuronal chloride transport, but certain plasticity mechanisms might be necessary to avoid sudden reversal of chloride homeostasis and over excitation. Neuronal activities dynamically modulate GABA_{A} receptor composition, intracellular trafficking, lateral mobility on neuronal surfaces, and synapse stability^{19,20}. SSADH restoration might lead to further reduction of GABA-mediated signaling in a setting of reduced GABA_{A} receptor availability, resulting in seizures. Adaptive changes (e.g., plasticity) in GABA receptors must be in place to accommodate loss of ambient GABA and loss of inhibitory tone, and avoid provoking seizures (FIG. 3). More broadly, GABA circuit maturation triggers critical period plasticity in the cortex²¹⁻²¹. Knock-down of critical factors for GABA circuit maturation delays critical period onset across brain regions²³⁻²⁵. It is unclear how critical period timing is affected in SSADHD, and whether such plasticity might represent an opportunistic window for successful therapy later in life. This is particularly relevant for adult SSADHD patients, whose critical period might have closed prematurely due to predominant GABA accumulation in early life. Subsequent lack of GABA_{A} receptor up-regulation upon SSADH restoration might render ERT ineffective. In this situation, adjunctive therapy targeted to re-open critical period plasticity²⁶ might become a realistic consideration along with ERT.

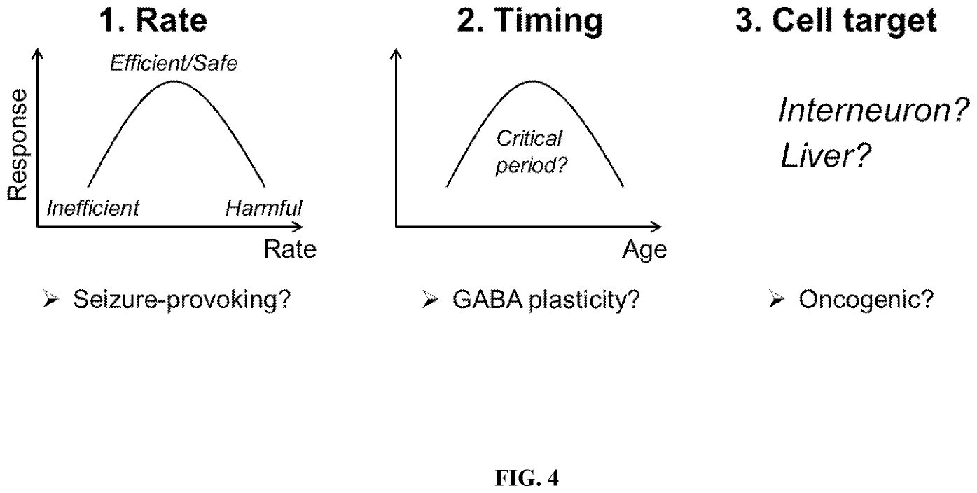

Described herein are three key parameters that, at least in part, can be used to identify ideal candidates for human clinical trials of SSADH gene therapy. As shown in FIG. 4, these three key parameters include (1) rate of restoration, (2) age of treatment, and (3) cell targets. The novel SSADHD mouse model described herein was used to generate relevant data that can be used to guide selection of appropriate viral vector candidates for clinical trials. For example, when the rate of restoration cannot exceed a certain threshold (otherwise causing seizures and brain injury), viral vectors that allow repeated administration of smaller doses with low immunogenicity will be therapeutically beneficial^{58,59}. In another example, when restoration should be targeted to specific cell types, viral vectors that allow the incorporation of cell-specific regulatory promoter elements can be considered⁶⁰.

Accordingly, the present disclosure provides, in some aspects, polynucleotides for restoring SSADH and methods of use thereof for treating SSADHD.

### I. Polynucleotides Encoding SSADH

Aspects of the present disclosure provide vectors (e.g., viral vectors such as adenoviral vectors, lentiviral vectors, adeno-associated viral (AAV) vectors) comprising a polynucleotide sequence encoding SSADH (ALDH5A1 encodes SSADH). In some embodiments, the polynucleotide sequence encoding SSADH is naturally occurring, e.g., the polynucleotide sequence of ALDH5A1 provided in SEQ ID NO:1.

ALDH5A1 cDNA (1608 bp):
(SEQ ID NO: 1)
ATGGCTACATGTATTTGGCTTCGAAGTTGTGGAGCTCGACGACTTGGATCTACATTTCCAGGATGTCGA
CTTCGACCTCGAGCTGGAGGACTTGTTCCTGCTTCTGGACCTGCTCCTGGACCTGCTCAACTTCGATGTTATGCT
GGACGACTTGCTGGACTTTCTGCTGCACTTCTTCGAACAGATAGTTTTGTTGGAGGACGATGGCTTCCTGCTGCT
GCAACATTTCCTGTTCAAGATCCTGCTAGTGGAGCTGCTCTTGGAATGGTAGCTGATTGTGGAGTTCGAGAAGCT
CGAGCTGCTGTTCGAGCTGCTTATGAAGCATTCTGCCGATGGAGAGAAGTCTCCGCCAAGGAGAGGAGTTCATTA
CTTCGGAAGTGGTACAATTTAATGATACAAAATAAGGATGACCTTGCCAGAATAATCACAGCTGAAAGTGGAAAG
CCACTGAAGGAGGCACATGGAGAAATTCTCTATTCCGCCTTTTTCCTAGAGTGGTTCTCTGAGGAAGCCCGCCGT
GTTTACGGAGACATTATCCACACCCCGGCAAAGGACAGGCGGGCCCTGGTCCTCAAGCAGCCCATAGGCGTGGCT
GCAGTCATCACCCCGTGGAATTTCCCCAGTGCCATGATCACCCGGAAGGTGGGGGCCGCCCTGGCAGCCGGCTGT
ACTGTCGTGGTGAAGCCTGCCGAAGACACGCCCTTCTCCGCCCTGGCCCTGGCTGAGCTTGCAAGCCAGGCTGGG
ATTCCTTCAGGTGTATACAATGTTATTCCCTGTTCTCGAAAGAATGCCAAGGAAGTAGGGGAGGCAATTTGTACT
GATCCTCTGGTGTCCAAAATTTCCTTTACTGGTTCAACAACTACAGGAAAGATCCTGTTGCACCACGCAGCAAAC
TCTGTGAAAAGGGTCTCTATGGAGCTGGGCGGCCTTGCTCCATTTATAGTATTTGACAGTGCCAACGTGGACCAG
GCTGTAGCAGGGGCCATGGCATCTAAATTTAGGAACACTGGACAGACTTGTGTTTGCTCAAACCAATTCTTGGTG
CAAAGGGGCATCCATGATGCCTTTGTAAAAGCATTCGCCGAGGCCATGAAGAAGAACCTGCGCGTAGGTAATGGA
TTTGAGGAAGGAACTACTCAGGGCCCATTAATTAATGAAAAAGCGGTAGAAAAGGTGGAGAAACAGGTGAATGAT
GCCGTTTCTAAAGGTGCCACCGTTGTGACAGGTGGAAAACGACACCAACTTGGAAAAAATTTCTTTGAGCCTACC
CTGCTGTGCAATGTCACCCAGGACATGCTGTGCACTCATGAAGAGACTTTCGGGCCTCTGGCACCAGTTATCAAG
TTCGATACAGAGGAGGAGGCTATAGCAATCGCTAACGCAGCTGATGTTGGGTTAGCAGGTTATTTTTACTCTCAA
GACCCAGCCCAGATCTGGAGAGTGGCAGAGCAGCTGGAAGTGGGCATGGTTGGCGTCAACGAAGGATTAATTTCC
TCTGTGGAGTGCCCTTTTGGTGGAGTGAAGCAGTCCGGCCTTGGGCGAGAGGGGTCCAAGTATGGCATTGATGAG
TATCTGGAACTCAAGTATGTGTGTTACGGGGGCTTGTAG

In some embodiments, the polynucleotide sequence encoding SSADH comprises a nucleotide sequence having at least 90%, at least 95%, at least 98%, at least 99% or 100% identity to SEQ ID NO:1.

To determine the percent identity of two amino acid sequences, the sequences are aligned for optimal comparison purposes (e.g., gaps can be introduced in one or both of a first and a second amino acid for optimal alignment and non-homologous sequences can be disregarded for comparison purposes). When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

Comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Also within the scope of the present disclosure are polynucleotides encoding a variant of SSADH. As used herein, the term “variant” refers to a nucleic acid having characteristics that deviate from what occurs in nature, e.g., a “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type nucleic acid. For example, a SSADH variant can be encoded by a polynucleotide sequence having at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to SEQ ID NO:1.

In some embodiments, the polynucleotide sequence encoding a variant of SSADH comprises one or more substitutions as compared to the wild type sequence. The one or more substitutions can be silent, i.e., they do not modify the amino acid sequence of any encoded protein (or otherwise result in a variant amino acid sequence). Alternatively, the one or more substitutions can result in modifications to the amino acid sequence of SSADH, resulting in an encoded protein having one or more amino acid substitutions (e.g., having 1, 2, 3, 4, 5, 6, 7, 8, 9, 10, 10-15, or 15-20 amino acid substitutions) relative to the wild type protein sequence. In some embodiments, a SSADH variant includes a chemical modification and/or a truncation. In some embodiments, a SSADH protein having one or more amino acid substitutions retains wild type protein function, or retains substantially the same function (e.g., at least 25%, at least 50%, at least 75%, e.g., 50-75%, or 75-100% of the function) as the wild type protein function. The term variant encompasses functional fragments of a wild type nucleic acid sequence.

II. Recombinant AAV (rAAV) Vectors and Particles

Aspects of the present disclosure provided rAAV vectors comprising a nucleotide sequence encoding SSADH that can be used for gene therapy for SSADHD. As used herein, the term “vector” can refer to a nucleic acid vector (e.g., a plasmid or recombinant viral genome), a wild-type AAV genome, or a virus that comprises a viral genome.

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners. Either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. A mature AAV capsid is composed of VP1, VP2, and VP3 (molecular masses of approximately 87, 73, and 62 kDa respectively) in a ratio of about 1:1:10.

A recombinant nucleic acid vector (hereafter referred to as a “rAAV vector”) can comprise a nucleotide sequence encoding SSADH; and one or more regions comprising sequences that facilitate the integration of the nucleotide sequence encoding SSADH (optionally with the one or more nucleic acid regions comprising a sequence that facilitates expression) into the genome of the subject. In some embodiments, the sequences facilitating the integration of the nucleotide sequence encoding SSADH (optionally with the one or more nucleic acid regions comprising a sequence that facilitates expression) into the genome of the subject are inverted terminal repeat (ITR) sequences (e.g., wild-type ITR sequences or engineered ITR sequences) flanking the nucleotide sequence encoding SSADH. The ITR sequences can be derived from any AAV serotype or can be derived from more than one serotype or pseudotyped. In some embodiments, the ITR sequences are derived from AAV9 serotype. In some embodiments, the ITR sequences are the same serotype as the capsid (e.g., AAV9 ITR sequences and AAV9 capsid). In some embodiments, the ITR sequences are derived from AAV-PHP.B or AAV-PHP.eB serotype. ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Calif.; and Addgene, Cambridge, Mass.; and Curtis A. Machida. Methods in Molecular Medicine™. Viral Vectors for Gene Therapy Methods and Protocols. © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, rAAV vectors can comprise one or more regulatory elements. Non-limiting examples of regulatory elements include promoters, insulators, silencers, response elements, introns, enhancers, initiation sites, internal ribosome entry sites (IRES) termination signals, and poly(A) signals. Any combination of such regulatory elements is contemplated herein (e.g., a promoter and a poly(A) signal).

In some embodiments, the rAAV vectors comprise a promoter that is operably linked to the coding sequence of the nucleotide sequence encoding SSADH. The term “promoter,” as used herein, refers to a control region of a nucleic acid at which initiation and rate of transcription of the remainder of a nucleic acid sequence are controlled. A promoter drives transcription of the nucleic acid sequence that it regulates, thus, it is typically located at or near the transcriptional start site of a gene. A promoter may have, for example, a length of 100 to 2000 nucleotides or a length of 100 to 3000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence.

In some embodiments, the promoter is a cell-type-specific promoter that restricts expression of SSADH to specific cell types, e.g., a neuron-specific promoter that restricts expression of SSADH to neurons. Non-limiting examples of promoters for use in rAAV vectors described herein include a γ-aminobutyric acid (GABA) transporter 1 (GAT1) promoter sequence, a GABA transporter 3 (GAT3) promoter sequence, a 5-hydroxytryptamin receptor (5HT3R) promoter sequence, a somatostatin (SST) promoter sequence, a parvalbumin (PV) promoter sequence, a Ca²⁺/calmodulin-dependent kinase subunit α (CaMKII) promoter sequence, neuron-specific enolase (NSE) promoter sequence, synapsin I with a minimal CMV sequence (Syn I-minCMV) promoter sequence, glial fibrillary acidic protein (GFAP) promoter sequence, an astrocyte promoter sequence, an aldehyde dehydrogenase 5 family member A1 (ALDH5A1) promoter sequence, or a combination thereof.

In some embodiments, the promoter comprises a ALDH5A1 promoter, e.g., the ALDH5A1 promoter sequence (referred to as FLnP sequence) provided in SEQ ID NO:2.

FLnP sequence (1850 bp):
(SEQ ID NO: 2)
TGGCTTTTCTGGGCCTCAGTTGATTGAGGCCTCAATTGAATGATTTCCCCAGGCCAGTAGAAATGAGCCCTGTAA
TCCCAGCACTTTGGGAGGCCAAGGAGGGCGGATAACTTGAGGTCAGGCATTCAAGACCAGCCTGGCCAACACGGT
GAAAACCTGTCTCTACTAAAAATACAAAAATTAGCTGGGCTTGGTGGCACACACCTGTAGCCCCATCTACTCGGG
AGGCTGAGGCAGGAGAATCGCTTGAACCCGGGAGGCGGAAGTTGCAGGGAGCTGAAATCGCACCACTGCACTCCA
CCCTGGGTGACAGAATGAGTCTGTCGGTCTCACAAGAACAAAACAAACAAAAAACAAAAACCACCACCAAAAACA
AAACAAAACAAAAACAAAAACTCCCTCCGCAGGCAGGAGATGTAAACACGCCAGCTCCCCAGCCCCACCTGTGAT
ACCTACTGGCCCACATCCCTATTAGGACTCCCTGGTTCTCCCCAGTTCCTGCAATAAAATACAGAGCAAAATAGT
GTGAAATTGATTAAACTCTACTGTTATCACTTCTGCCATATGTGACTGGCTCAAAACTCAATGGATCTGACTCAG
CAAAAGACAAAGTCAAAATTTAAAGTTACTGCCTCGGCATTTTGCAAATGTCCTAGAAAAAAATTCAACGTCCTA
GAAAAAGGTGAAAGCTAGCAAGATACTCTCAACGCTGTAGGAGTTGAGAAAGGGAGATATGTAACCTAAAATCAA
TGCATAGGGCTGGGCTTCATTTGCACCCACAAATTCCCAGCATACGCCTGCTATTTTTATTTTTATCCTCATTAG
GTTTTAAGCTTCCCTGCTGCTTTCAGAGTCTTAAGCCCAAGTCTAATTTTTTAACGGATGAAGAATGCTGTTTAA
AGGTGCTCAAATACCTTGGTTGGATAGTATGCTCAATCCTTATACCTTGGTTGATTCAATTTTGACAAATGCTTC
CATTTACTAGTTGGTGACCCAACTTTAATCTTACTAGTGGCTTGACTAGGGTAGCTAGTATGACTTTGAACGCTA
TATTGAATTTAATGTGGCAACTGTAATGAGCATTTTACGTTATCTCACAACAAAATGGGGTAGATATGGTTCTAT
TTTATAAGTGACTGGACTAAGGTTAACCTGTCGGTCACACAGCTTGTCGGCAACAGACTTGAGTTTGGATCCTAG
TCTGTTTATTTCCAAAGCGCTGTTATTTATCGGGAGCAACCCTAGGAGAATGCCTAGACACACACCCAAAAAAGC
AGCCAGGCAGCAGAACGCGGGGTCACACTTCGACCCCTCAGAGAACGATCGCTCCCAATCAATACTACGTGCTCA
GGAGCTACAACTGAAGAAGCGTGATCACGGCCTTGGCATTTAGGGCAGGCACCAGCGCATCTATGGACCGCGCAC
AAATTCCGGGGATGCCGAATTTGGGGGATGCTAAGGGGAGAGAGTGGGTCTCTAGCAGCGATTGGGGGCTCAGGA
GCAGTTAGTGACAAATGAGCACCCGAAAAGTGAAAAGGTGACAGCAGTCCGCAGGTGCATCTACTGGCGAGCCTT
CTCCATCCCCGAACCCAACCCTCCCCCGGGAGAAGGTCGCGCCAGGAGAGAAGCCGCGCGGCGCTTAGGGCAAGG
TGCAGAGGGCGGCGCGGCGGTGCAGCGAGAAAGACGCGGAGAGAGGGCGCTGCTCTGTGGCTCTGCAACCTTCCG
CCAGCTCCCACGCTTTCCCCGCGCGTCCCCGGCGCCTCCTCGCTCCTCTTGCTTCCCCGCGACCCCTGCGTTCCC
GTGCGCGCGCGCCCGCTTGCCTGTTTCCTGTCGCCGTCGTTGCCCGGGCC

In some embodiments, the promoter sequence comprises a sequence having at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to SEQ ID NO:2, that retains the ability to drive expression of an operably-linked gene in neuronal cells.

In some embodiments, the rAAV vector comprises a ALDH5A1 promoter operably linked to a nucleotide sequence encoding SSADH, e.g., the ALDH5A1 promoter sequence set forth in SEQ ID NO:2 operably linked to a nucleotide sequence encoding SSADH set forth in SEQ ID NO:1, which is referred to as FLnP-hALDH5A1 and is provided in SEQ ID NO:3.

FLnP-hALDH5A1 (3458 bp):
(SEQ ID NO: 3)
TGGCTTTTCTGGGCCTCAGTTGATTGAGGCCTCAATTGAATGATTTCCCCAGGCCAGTAGAAATGAGCC
CTGTAATCCCAGCACTTTGGGAGGCCAAGGAGGGCGGATAACTTGAGGTCAGGCATTCAAGACCAGCCTGGCCAA
CACGGTGAAAACCTGTCTCTACTAAAAATACAAAAATTAGCTGGGCTTGGTGGCACACACCTGTAGCCCCATCTA
CTCGGGAGGCTGAGGCAGGAGAATCGCTTGAACCCGGGAGGCGGAAGTTGCAGGGAGCTGAAATCGCACCACTGC
ACTCCACCCTGGGTGACAGAATGAGTCTGTCGGTCTCACAAGAACAAAACAAACAAAAAACAAAAACCACCACCA
AAAACAAAACAAAACAAAAACAAAAACTCCCTCCGCAGGCAGGAGATGTAAACACGCCAGCTCCCCAGCCCCACC
TGTGATACCTACTGGCCCACATCCCTATTAGGACTCCCTGGTTCTCCCCAGTTCCTGCAATAAAATACAGAGCAA
AATAGTGTGAAATTGATTAAACTCTACTGTTATCACTTCTGCCATATGTGACTGGCTCAAAACTCAATGGATCTG
ACTCAGCAAAAGACAAAGTCAAAATTTAAAGTTACTGCCTCGGCATTTTGCAAATGTCCTAGAAAAAAATTCAAC
GTCCTAGAAAAAGGTGAAAGCTAGCAAGATACTCTCAACGCTGTAGGAGTTGAGAAAGGGAGATATGTAACCTAA
AATCAATGCATAGGGCTGGGCTTCATTTGCACCCACAAATTCCCAGCATACGCCTGCTATTTTTATTTTTATCCT
CATTAGGTTTTAAGCTTCCCTGCTGCTTTCAGAGTCTTAAGCCCAAGTCTAATTTTTTAACGGATGAAGAATGCT
GTTTAAAGGTGCTCAAATACCTTGGTTGGATAGTATGCTCAATCCTTATACCTTGGTTGATTCAATTTTGACAAA
TGCTTCCATTTACTAGTTGGTGACCCAACTTTAATCTTACTAGTGGCTTGACTAGGGTAGCTAGTATGACTTTGA
ACGCTATATTGAATTTAATGTGGCAACTGTAATGAGCATTTTACGTTATCTCACAACAAAATGGGGTAGATATGG
TTCTATTTTATAAGTGACTGGACTAAGGTTAACCTGTCGGTCACACAGCTTGTCGGCAACAGACTTGAGTTTGGA
TCCTAGTCTGTTTATTTCCAAAGCGCTGTTATTTATCGGGAGCAACCCTAGGAGAATGCCTAGACACACACCCAA
AAAAGCAGCCAGGCAGCAGAACGCGGGGTCACACTTCGACCCCTCAGAGAACGATCGCTCCCAATCAATACTACG
TGCTCAGGAGCTACAACTGAAGAAGCGTGATCACGGCCTTGGCATTTAGGGCAGGCACCAGCGCATCTATGGACC
GCGCACAAATTCCGGGGATGCCGAATTTGGGGGATGCTAAGGGGAGAGAGTGGGTCTCTAGCAGCGATTGGGGGC
TCAGGAGCAGTTAGTGACAAATGAGCACCCGAAAAGTGAAAAGGTGACAGCAGTCCGCAGGTGCATCTACTGGCG
AGCCTTCTCCATCCCCGAACCCAACCCTCCCCCGGGAGAAGGTCGCGCCAGGAGAGAAGCCGCGCGGCGCTTAGG
GCAAGGTGCAGAGGGCGGCGCGGCGGTGCAGCGAGAAAGACGCGGAGAGAGGGCGCTGCTCTGTGGCTCTGCAAC
CTTCCGCCAGCTCCCACGCTTTCCCCGCGCGTCCCCGGCGCCTCCTCGCTCCTCTTGCTTCCCCGCGACCCCTGC
GTTCCCGTGCGCGCGCGCCCGCTTGCCTGTTTCCTGTCGCCGTCGTTGCCCGGGCCATGGCTACATGTATTTGGC
TTCGAAGTTGTGGAGCTCGACGACTTGGATCTACATTTCCAGGATGTCGACTTCGACCTCGAGCTGGAGGACTTG
TTCCTGCTTCTGGACCTGCTCCTGGACCTGCTCAACTTCGATGTTATGCTGGACGACTTGCTGGACTTTCTGCTG
CACTTCTTCGAACAGATAGTTTTGTTGGAGGACGATGGCTTCCTGCTGCTGCAACATTTCCTGTTCAAGATCCTG
CTAGTGGAGCTGCTCTTGGAATGGTAGCTGATTGTGGAGTTCGAGAAGCTCGAGCTGCTGTTCGAGCTGCTTATG
AAGCATTCTGCCGATGGAGAGAAGTCTCCGCCAAGGAGAGGAGTTCATTACTTCGGAAGTGGTACAATTTAATGA
TACAAAATAAGGATGACCTTGCCAGAATAATCACAGCTGAAAGTGGAAAGCCACTGAAGGAGGCACATGGAGAAA
TTCTCTATTCCGCCTTTTTCCTAGAGTGGTTCTCTGAGGAAGCCCGCCGTGTTTACGGAGACATTATCCACACCC
CGGCAAAGGACAGGCGGGCCCTGGTCCTCAAGCAGCCCATAGGCGTGGCTGCAGTCATCACCCCGTGGAATTTCC
CCAGTGCCATGATCACCCGGAAGGTGGGGGCCGCCCTGGCAGCCGGCTGTACTGTCGTGGTGAAGCCTGCCGAAG
ACACGCCCTTCTCCGCCCTGGCCCTGGCTGAGCTTGCAAGCCAGGCTGGGATTCCTTCAGGTGTATACAATGTTA
TTCCCTGTTCTCGAAAGAATGCCAAGGAAGTAGGGGAGGCAATTTGTACTGATCCTCTGGTGTCCAAAATTTCCT
TTACTGGTTCAACAACTACAGGAAAGATCCTGTTGCACCACGCAGCAAACTCTGTGAAAAGGGTCTCTATGGAGC
TGGGCGGCCTTGCTCCATTTATAGTATTTGACAGTGCCAACGTGGACCAGGCTGTAGCAGGGGCCATGGCATCTA
AATTTAGGAACACTGGACAGACTTGTGTTTGCTCAAACCAATTCTTGGTGCAAAGGGGCATCCATGATGCCTTTG
TAAAAGCATTCGCCGAGGCCATGAAGAAGAACCTGCGCGTAGGTAATGGATTTGAGGAAGGAACTACTCAGGGCC
CATTAATTAATGAAAAAGCGGTAGAAAAGGTGGAGAAACAGGTGAATGATGCCGTTTCTAAAGGTGCCACCGTTG
TGACAGGTGGAAAACGACACCAACTTGGAAAAAATTTCTTTGAGCCTACCCTGCTGTGCAATGTCACCCAGGACA
TGCTGTGCACTCATGAAGAGACTTTCGGGCCTCTGGCACCAGTTATCAAGTTCGATACAGAGGAGGAGGCTATAG
CAATCGCTAACGCAGCTGATGTTGGGTTAGCAGGTTATTTTTACTCTCAAGACCCAGCCCAGATCTGGAGAGTGG
CAGAGCAGCTGGAAGTGGGCATGGTTGGCGTCAACGAAGGATTAATTTCCTCTGTGGAGTGCCCTTTTGGTGGAG
TGAAGCAGTCCGGCCTTGGGCGAGAGGGGTCCAAGTATGGCATTGATGAGTATCTGGAACTCAAGTATGTGTGTT
ACGGGGGCTTGTAG

In some embodiments, the rAAV vector comprises a FLnP-hALDH5A1 sequence having at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to SEQ ID NO:3.

In some embodiments, the rAAV vector comprises a polyadenylation (pA) signal. Eukaryotic mRNAs are typically transcribed as a precursor mRNA. The precursor mRNA is processed to generated the mature mRNA, including a polyadenylation process. The process of polyadenylation begins as the transcription of a gene terminates. The 3′-most segment of the newly-made precursor mRNA is first cleaved off by a set of proteins. These proteins then synthesize the poly(A) tail at the RNA's 3′ end. The cleavage site typically contains the polyadenylation signal, e.g., AAUAAA. The poly(A) tail is important for the nuclear export, translation, and stability of mRNA.

In some embodiments, the rAAV vector comprises at least, in order from 5′ to 3′, a first adeno-associated virus (AAV) inverted terminal repeat (ITR) sequence, a promoter operably linked to a nucleotide encoding SSADH, a polyadenylation signal, and a second AAV inverted terminal repeat (ITR) sequence.

The rAAV vector can be circular or linear. The rAAV can be single-stranded or double-stranded. In some embodiments, the rAAV vector is a self-complementary rAAV vector. Any rAAV vector described herein may be encapsidated by a viral capsid, such as an AAV9 capsid or variant thereof (PHP.B or PHP.eB) or any other serotype or variant thereof.

Aspects of the present disclosure provide rAAV particles or compositions comprising such particles. The rAAV particles comprise a viral capsid and an rAAV vector as described herein, which is encapsidated by the viral capsid. Methods of producing rAAV particles are known in the art and are commercially available (see, e.g., Zolotukhin et al. Production and purification of serotype 1, 2, and 5 recombinant adeno-associated viral vectors. Methods 28 (2002) 158-167; and U.S. Patent Application Publication Numbers US 2007/0015238 and US 2012/0322861, which are incorporated herein by reference; and plasmids and kits available from ATCC and Cell Biolabs, Inc.). For example, a plasmid containing the rAAV vector can be combined with one or more helper plasmids, e.g., that contain a rep gene (e.g., encoding Rep78, Rep68, Rep52 and Rep40) and a cap gene (e.g., encoding VP1, VP2, and VP3), and transfected into a producer cell line such that the rAAV particle can be packaged and subsequently purified.

The rAAV vectors or the rAAV particles can be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV vector or an rAAV particle refers to the serotype of the capsid proteins of the recombinant virus. In some embodiments, the rAAV particle is rAAV5. In some embodiments, the rAAV particle is rAAV9 or a derivative thereof such as AAV-PHP.B or AAV-PHP.eB. Non-limiting examples of derivatives and pseudotypes include AAVrh.10, rAAV2/1, rAAV2/5, rAAV2/8, rAAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y→F), AAV8 (Y733F), AAV2.15, AAV2.4, AAVM41, and AAVr3.45. AAV serotypes and derivatives/pseudotypes, and methods of producing such are known in the art (see, e.g., Mol Ther. 2012 April; 20(4):699-708). In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Other viral vectors can be used to deliver nucleic acids encoding SSADH to a cell. Such viral vectors include, but are not limited to, lentivirus vectors, alphavirus vectors, enterovirus vectors, pestivirus vectors, baculovirus vectors, herpesvirus vectors, Epstein Barr virus vectors, papovavirus vectors, poxvirus vectors, vaccinia virus vectors, and herpes simplex virus vectors.

Any vehicle suitable for delivery of nucleic acids encoding SSADH to a cell can be used in methods described herein. For example, nucleic acids encoding SSADH can be delivered to a cell using non-viral nucleic acid encapsulations technologies (e.g., lipid nanoparticles) or virus capsid protein-based vehicles (e.g., Simian virus 40 major capsid protein-based vehicles).

III. rAAV Gene Therapy for SSADHD

Provided herein are methods for treating SSADHD using the rAAV vectors, the rAAV particles comprising the rAAV vectors, or compositions comprising the rAAV particles of the present disclosure. In some embodiments, methods for treating SSADHD involve restoring, at least in part, expression and/or activity of SSADH using the rAAV vectors, the rAAV particles comprising the rAAV vectors, or the compositions comprising the rAAV particles.

As used herein, “SSADHD” refers to a rare autosomal recessive neurologic disorder in which an enzyme defect in the GABA degradation pathway causes a consecutive elevation of gamma-hydroxybutyric acid (GHB) and GABA. In some embodiments, the enzyme defect in the GABA degradation pathway comprises a defect in expression and/or activity of SSADH.

To practice the method disclosed herein, an effective amount of the rAAV vectors, the rAAV particles comprising the rAAV vectors, or the compositions comprising the rAAV particles can be administered to a subject having or at risk for having SSADH via a suitable route.

The term “subject” refers to a subject who needs treatment as described herein. In some embodiments, the subject is a human (e.g., a human patient) or a non-human mammal (e.g., mouse, rat, cat, dog, horse, cow, goat, or sheep). A human subject who needs treatment can be a human patient having, suspected of having, or at risk for having SSADHD. A subject having SSADHD can be identified by routine medical examination, e.g., medical examination (e.g., history and physical), or laboratory tests (e.g., urinalysis for high levels of GHB). Such a subject can exhibit one or more symptoms associated with SSADHD, e.g., delayed gross motor development, delayed mental development, autism, attention deficit, delayed fine motor skill development, delayed speech and language development, hypotonia, epilepsy, hyporeflexia, ataxia, behavioral problems, hyperkinesis, or a combination thereof. Alternatively, or in addition to, such a subject can have one or more risk factors for SSADHD, e.g., genetic susceptibility and/or family history.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient can insist upon a lower dose or tolerable dose for medical reasons, psychological reasons, or virtually any other reason.

As used herein, the term “treating” refers to administration of a composition including one or more active agents to a subject who has SSADHD, a symptom of SSADHD, and/or a predisposition toward SSADHD, with the purpose to alleviate, relieve, alter, remedy, ameliorate, improve, or affect SSADHD and/or, a symptom of SSADHD. The present methods can also be used to reduce risk of developing SSADHD.

Alleviating SSADHD includes delaying the development or progression of the disease, and/or reducing disease severity. Alleviating the disease does not necessarily require curative results.

As used herein, “delaying” the development of SSADHD means to defer, hinder, slow, retard, stabilize, and/or postpone progression of SSADHD. This delay can be of varying lengths of time, depending on the history of SSADHD, and/or individuals being treated. A method that “delays” or alleviates the development of SSADHD and/or delays the onset of SSADHD is a method that reduces probability of developing one or more symptoms of SSADHD in a given time frame and/or reduces extent of the symptoms in a given time frame, when compared to not using the method. Such comparisons are typically based on clinical studies, using a number of subjects sufficient to give a statistically significant result.

“Development” or “progression” of SSADHD means initial manifestations and/or ensuing progression (worsening of symptoms or severity) of SSADHD. Development of SSADHD can be detectable and assessed using clinical techniques known in the art.

However, development also refers to progression that can be undetectable. For purposes of this disclosure, development or progression refers to the biological course of the symptoms. “Development” includes occurrence, recurrence, and onset. As used herein, “onset” or “occurrence” of SSADHD includes initial onset and/or recurrence.

In some embodiments, rAAV particles and/or rAAV vectors are administered to a subject in an amount sufficient to increase activity and/or expression of SSADH by at least 10% (e.g., at least 20%, at least 30%, at least 40%, at least 50%, at least 60%, at least 70%, at least 80%, at least 90%, or more).

In some embodiments, rAAV particles and/or rAAV vectors are administered to a subject in an amount sufficient to increase activity and/or expression of GABA receptors (e.g., GABA_{A} receptors) by at least 10% (e.g., at least 20%, at least 30%, at least 40%, at least 50%, at least 60%, at least 70%, at least 80%, at least 90%, or more).

rAAV particles and/or rAAV vectors can be delivered in the form of a composition, such as a composition comprising rAAV particles and/or rAAV vectors described herein, and a pharmaceutically acceptable carrier as described herein. rAAV particles and/or rAAV vectors can be prepared in a variety of compositions, and can also be formulated in appropriate pharmaceutical vehicles for administration to human or animal subjects.

rAAV particles administered to a subject can be provided in a composition having a concentration on the order ranging from 10⁶ to 10¹⁴ particles/ml or 10³ to 10¹⁵ particles/ml, or any values there between for either range, such as for example, about 10⁶, 10⁷, 10⁸, 10⁹, 10¹⁰, 10¹¹, 10¹², 10¹³, or 10¹⁴ particles/ml. In some embodiments, rAAV particles of higher than 10¹³ particles/ml are be administered. In some embodiments, the number of rAAV particles administered to a subject can be on the order ranging from 10⁶ to 10¹⁴ vector genomes (vgs)/ml or 10³ to 10¹⁵ vgs/ml, or any values there between for either range, such as for example, about 10⁶, 10⁷, 10⁸, 10⁹, 10¹⁰, 10¹¹, 10¹², 10¹³, or 10¹⁴ vgs/ml. In one embodiment, AAV particles of higher than 10¹³ vgs/ml are be administered.

rAAV particles and/or rAAV vectors can be administered as a single dose, or divided into two or more administrations as can be required to achieve partial or complete SSADH restoration. When administering multiple doses, the amount of rAAV particles and/or rAAV vectors in each dose can be the same or different. In some embodiments, the number of rAAV particles administered to a subject can be on the order ranging from 10⁶-10¹¹ vg/kg, or any values therebetween, such as for example, about 10⁶, 10⁷, 10¹, 10¹, 10¹⁰, 10¹¹, 10¹², 10¹³, or 10¹⁴ vgs/kg. In some embodiments, 0.0001 ml to 10 ml are delivered to a subject.

rAAV particles and/or rAAV vectors can be administered alone or as part of a combination therapy comprising an additional therapeutic agent. Any therapeutic agent suitable for treating SSADHD can be used as an additional therapeutic agent in methods and/or compositions described herein. Non-limiting examples of additional therapeutic agents include vigabatrin, sodium valproate, GABA_{B} receptor antagonists (e.g., CGP-35348), GABA_{B} agonists (e.g., baclofen), taurine, anticonvulsant drugs (e.g., ethosuximide), or combinations thereof. Alternatively, in some embodiments, no other agents are used in the methods described herein.

rAAV particles and/or rAAV vectors in suitably formulated pharmaceutical compositions disclosed herein can be administered either subcutaneously, parenterally, intravenously, intramuscularly, intraperitoneally, by oral or nasal inhalation, or by direct injection to one or more cells, tissues, fluid (e.g., cerebrospinal fluid) or organs (e.g., brain). In some embodiments, the administration is a route suitable for systemic delivery, such as by intravenous injection or infusion. In some embodiments, the administration is to the central nervous system, e.g., via intracerebroventricular injection or intrathecal injection.

Pharmaceutical compositions comprising rAAV particles and/or rAAV vectors described herein can be suitable for injectable use include sterile aqueous solutions or dispersions. In some embodiments, the pharmaceutical composition is stable under the conditions of manufacture and storage and is preserved against the contaminating action of microorganisms, such as bacteria and fungi. The pharmaceutical composition can include a carrier, which can be a solvent or dispersion medium containing, for example, water, saline, ethanol, polyol (e.g., glycerol, propylene glycol, and liquid polyethylene glycol, and the like), suitable mixtures thereof, and/or vegetable oils. In some embodiments, proper fluidity can be maintained, for example, by the use of a coating, such as lecithin, by the maintenance of the required particle size in the case of dispersion and by the use of surfactants.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described can be more fully understood, the following examples are set forth. The examples described in this application are offered to illustrate the methods and compositions provided herein and are not to be construed in any way as limiting their scope.

### Materials and Methods

The following materials and methods were used in the Examples set forth herein.

### Institutional Assurance of Animal and Virus Use

All animal treatment procedures and viral materials described in this study were covered by protocols approved by the Institutional Animal Care and Use Committee (IACUC) and the Institutional Biosafety Committee (IBC) at Boston Children's Hospital.

### AAV Injection Into C57Bl/6 Mice

AAV-PHP.B:CAG-GFP (2.36×10¹³ gc/ml) was pre-packaged and obtained from the Viral Core of Boston Children's Hospital. AAV was suspended in sterile physiological saline and was administered into C57Bl/6 mice via intraperitoneal (IP) injection at post-natal day 10 (P10). Injections were performed once or across multiple days as indicated herein.

### Immunofluorescence Staining

Perfusion of cortical tissue and immunostaining procedures were performed as described previously³³. Under deep anesthesia, mice were perfused transcardially with ice-cold phosphate buffered saline (PBS) followed by 4% paraformaldehyde (PFA). Brain tissues were harvested, post-fixed in 4% PFA, and cryopreserved in Tissue-Plus OCT Compound (Fisher Healthcare, Waltham MA) for at least 24 hours before sectioning. Free-floating cryosections covering the hippocampus and the cerebellum (sagittal, 30 μm, mid-line ±1.2-1.7 mm³⁴⁻³⁶) were obtained at −20° C., washed briefly with PBS, incubated with primary antibodies overnight at 4° C., washed again, incubated with Alexa Fluor 594-conjugated secondary antibodies for 1 hour at room temperature, then mounted on glass slides. All perfusion, tissue fixation, and immunostaining procedures were carried out under the same conditions using the same batch of buffers to minimize variability between samples.

### Antibodies

Different primary antibodies against specific interneuron subclass are used in this study: Calretinin, vasoactive-intestinal polypeptide (VIP) and parvalbumin (PV). These interneuron subtypes show differential expression with brain region specificity.

### Image Acquisition

Immunostained brain sections were identified by fluorescence imaging under low power magnification (×10 objective). Image acquisition were carried out using the FV10-ASW software (v2.1 C), with the following parameters: 20% laser output, ×1 gain control, laser intensity between 500 and 700, offset between 10% and 15% such that signals were within the linear range. Individual channels were acquired sequentially. Confocal images under low power (10× objective) and high power (40× objective) were acquired in selected brain regions. The amount of AAV-mediated transgene expression was quantified by confocal imaging, represented by GFP intensity in arbitrary units (a.u.).

### Statistics

GFP intensity values from confocal imaging (represented by arbitrary units) were compared across experimental groups (e.g., across 1, 3 or 5 days of AAV injection) at two different post-injection time points (e.g., 7 days or 14 days). One-way ANOVA was used to compare across groups, followed by post-hoc Bonferroni's Multiple Comparison Test for statistical significance. Data from two independent experiments were combined.

### Example 1: Construction of a Novel SSADHD Mouse Model, aldh5a1^{STOP/STOP}

Described herein is a novel SSADHD mouse model that allows ‘on-demand’ SSADH restoration. In this design, we genetically engineered a gene cassette inactivating the endogenous aldh5a1 gene in mice, mimicking the human SSADHD disorder.

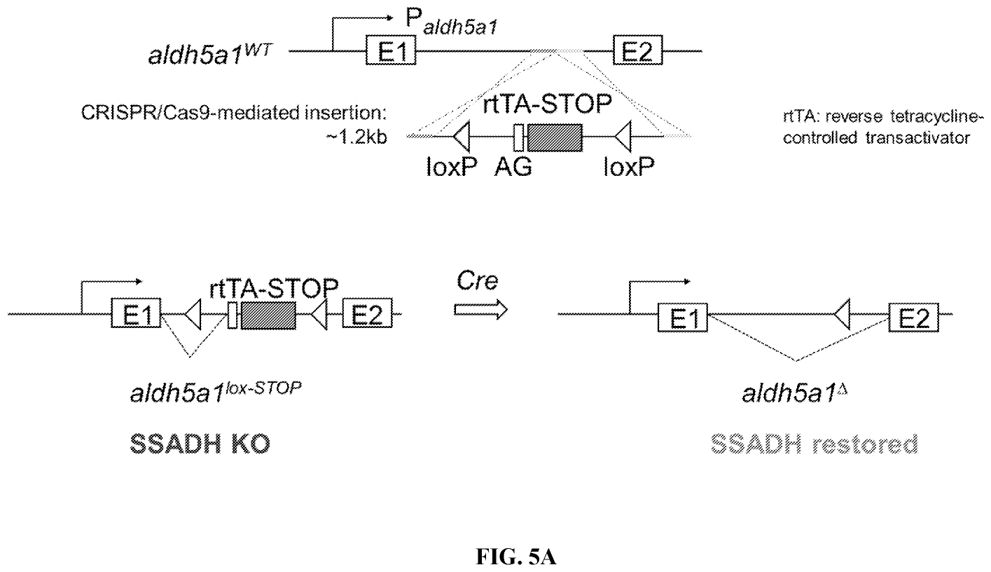

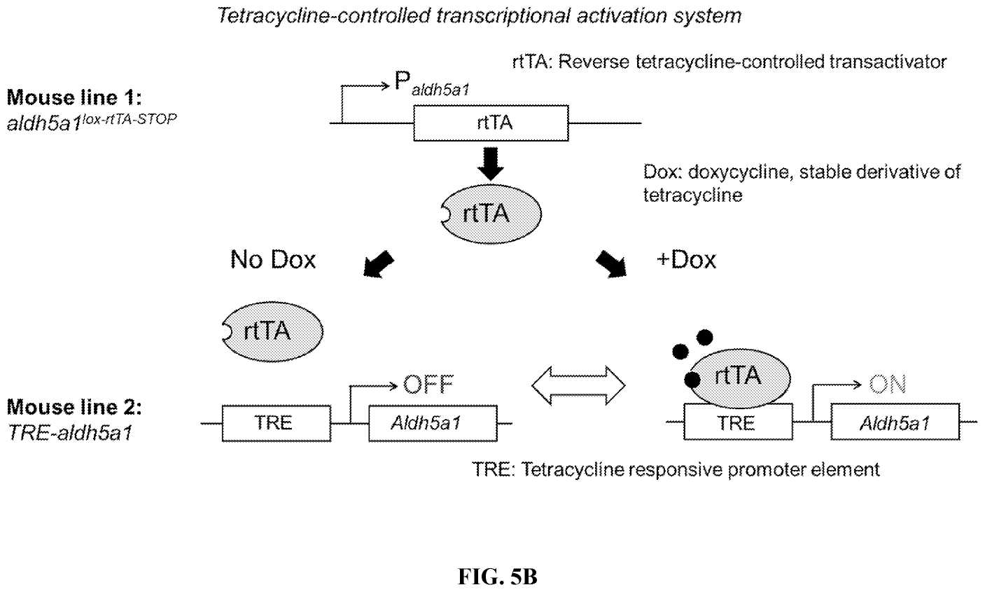

To construct the aldh5a1^{lox-rtTA-STOP} mouse, the endogenous aldh5a1 gene was disrupted by CRISPR/Cas9-mediated homology directed repair in its first intron with the insertion of a gene cassette containing a splice acceptor (AG) and the rtTA-STOP sequence flanked by two loxP sites (FIG. 5A, top panel). At baseline, aldh5a1^{lox-rtTA-STOP} mice are SSADH-null due to the disrupted aldh5a1 gene (FIG. 5A, middle panel). Instead, rtTA expression is driven by endogenous aldh5a1 promoter activities to combine with a second mouse, TRE-aldh5a1, for doxycycline-mediated rescue strategy (FIG. 5A, middle panel). Upon Cre-recombination, aldh5a1 is reconstituted for re-expression (aldh5a1^{Δ}) (FIG. 5A, bottom panel). Breeding aldh5a1^{lox-rtTA-STOP} and TRE-aldh5a1 mice allows reversible expression of recombinant aldh5a1 in the presence of doxycycline (Dox) tightly driven by a Tet-responsive element (TRE) (FIG. 5B).

### Example 2: Experimental Paradigms for Studying the Impacts of Rate and Age of SSADH Restoration in Mice

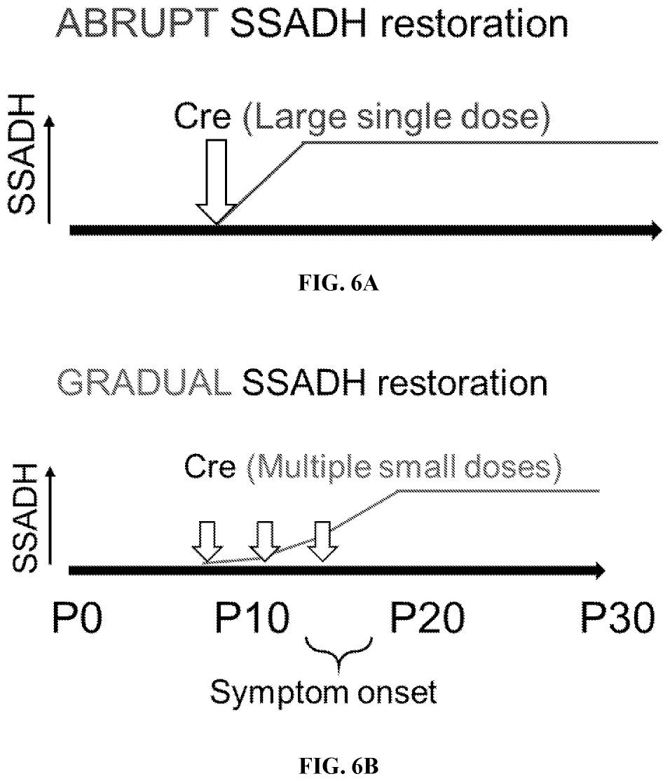

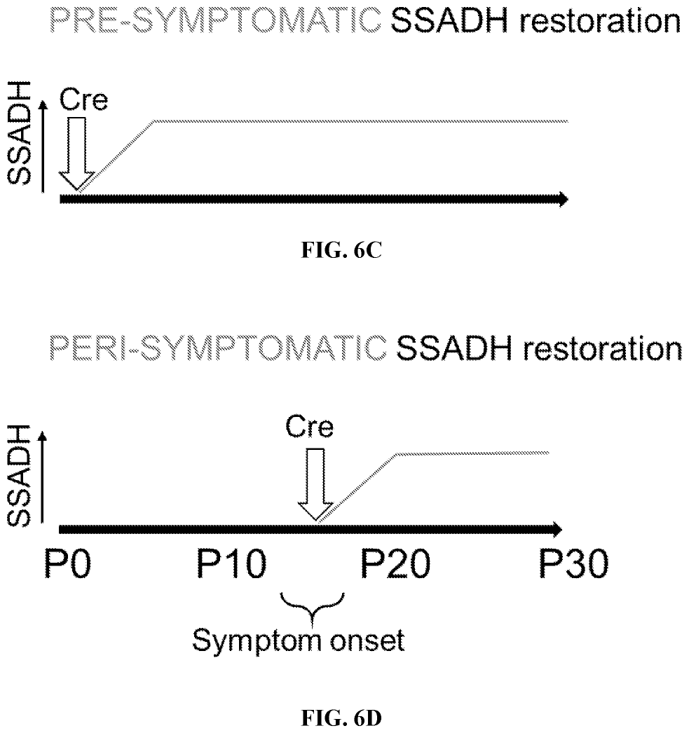

If SSADH restoration leads to ambient GABA reduction, then a safe rate of enzyme restoration will be determined by the maximum rate at which GABA (e.g., GABA_{A}) receptors are upregulated. Without wishing to be bound by theory, abrupt SSADH restoration can correspond to abrupt GABA decline without accompanying increase in GABA receptor expression that can lead to seizures and brain injury. In contrast, gradual SSADH replacement can enable compensatory GABA receptor upregulation, and can be better tolerated. Using the novel mouse model described herein, we will be able to test the safety and efficacy of a range of rates of enzyme restoration in SSADHD, and will explicitly address rate, rather than dose, as these pertain to gene therapy for epilepsy (FIGS. 6A-6B).

Given tight developmental regulation of GABAergic signaling, it is unclear whether SSADH restoration safe and effective across all ages. SSADH restoration can be safe and effective during specific developmental windows. GABA circuit plasticity is heightened during early critical periods of brain development^{21,30}. If successful SSADH restoration requires GABA circuit (e.g., GABA receptor) auto regulation to accommodate a profound decline in GABA concentration, then such therapy might only be effective in younger patients. Conversely, in older patients who lack GABA circuit plasticity, SSADH restoration might be ineffective and unsafe. This too requires explicit preclinical testing (FIGS. 6C-6D).

### Example 3: Rate-Dependent Transgene Expression in Brain Via AAV-PHP.B Systemic Injections

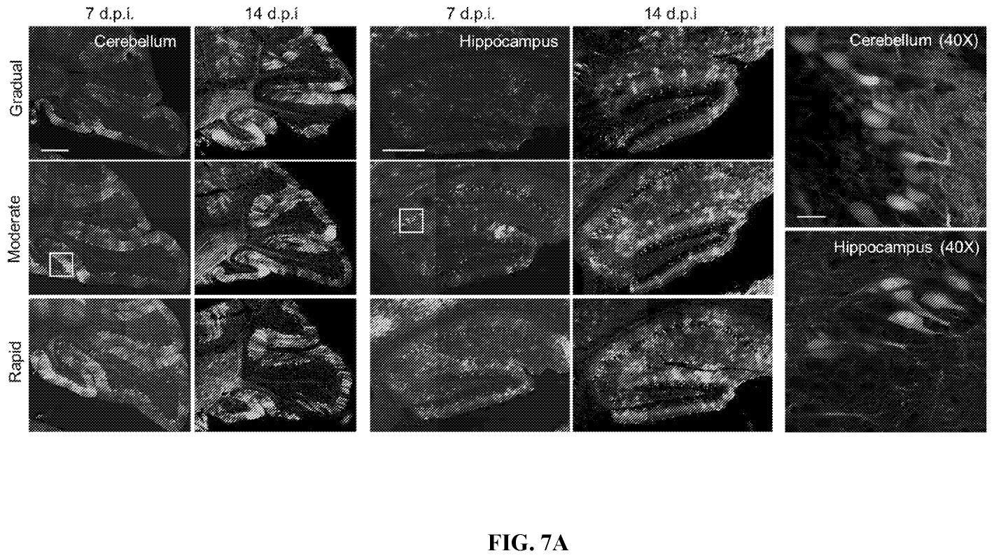

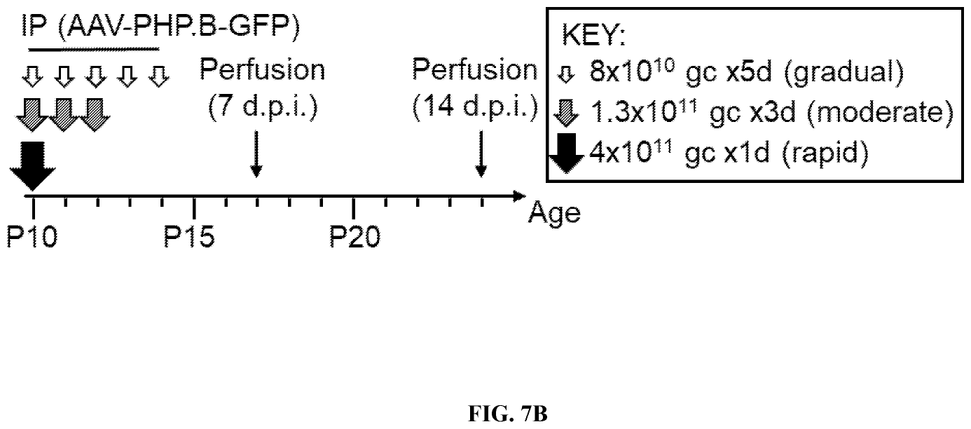

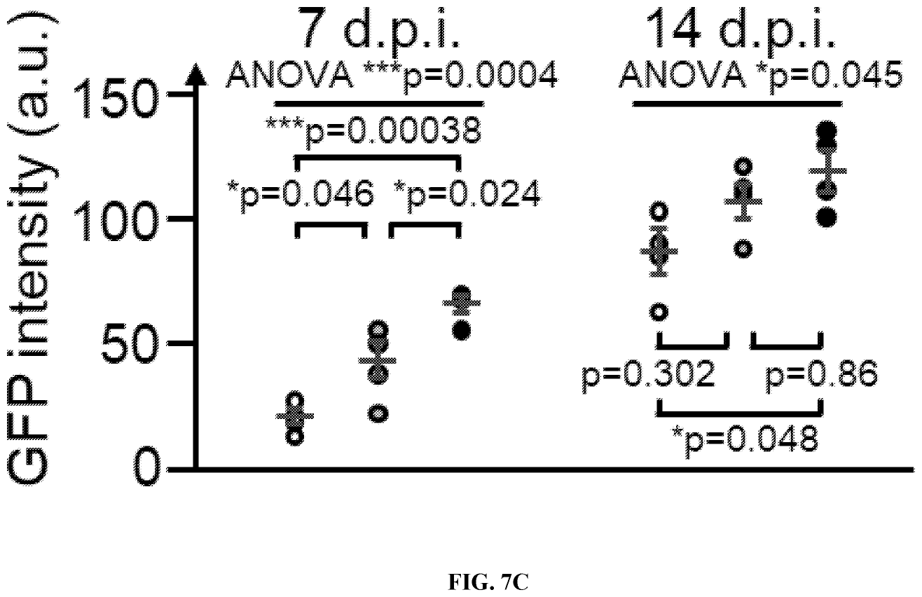

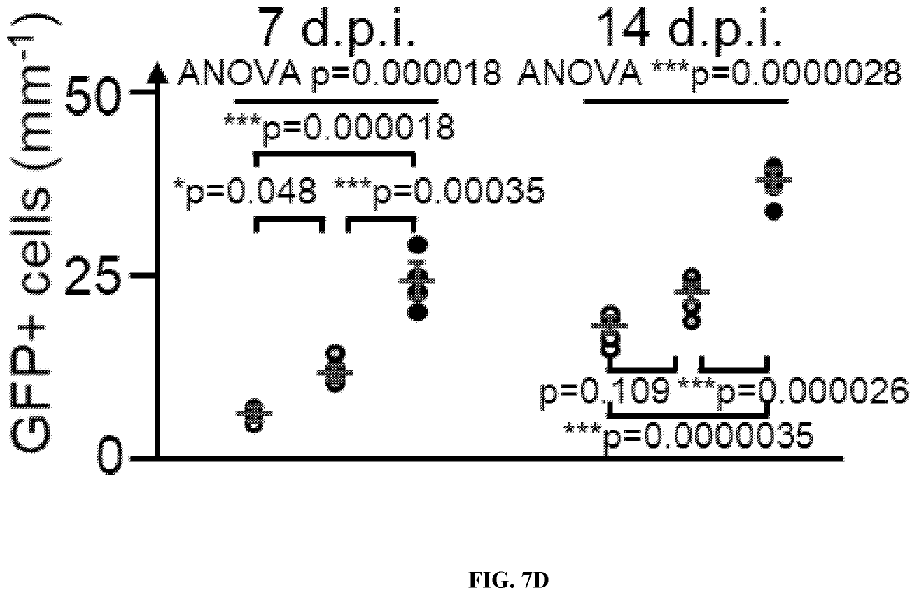

A proof-of-concept study was conducted to establish experimental paradigms for various rates of transgene expression via AAV vectors. Using an AAV construct which expresses GFP under constitutively active promoter (AAV-PHP.B:CAG-GFP, which is also referred to as AAV-GFP), we found that transgene expression is directly proportional to the rate of virus vector injection. AAV-PHP.B is an adeno-associated virus encapsulated with a blood-brain barrier penetrating capsid. A pilot study was performed where identical viral loads were delivered at once, or in 3-5 divided daily doses. We administered AAV-GFP via IP injection in C57Bl/6 mice on postnatal day 10 (P10), and quantified AAV transduction efficiency by confocal imaging on perfused brain slices at 7- or 14-day post injection (d.p.i.) (FIG. 7A). Using this injection paradigm (FIG. 7B), we observed widespread GFP expression in the brain, including the hippocampus and the cerebellum, which are relevant sites of robust SSADH expression. Importantly, our dosing strategies yielded >3-fold differential rates of gene expression in terms of GFP intensity at 7 d.p.i. (gradual=20.53±2.83 a.u; moderate=41.80±7.48 a.u.; rapid=66.03±3.47 a.u.) but the cumulative GFP intensity at 14 d.p.i. was largely diminished to <1.4-fold across groups (gradual=87.05±8.47 a.u.; moderate=107.2±6.86; rapid=119.7±7.96) (FIG. 7C). The number of GFP+ cells was greater at 14 d.p.i. compared to the number of GFP+ cells at 7 d.p.i (FIG. 7D).

### Example 4: AAV-PHP.B Transduces Interneuron Subtypes in the Hippocampus and the Cerebellum

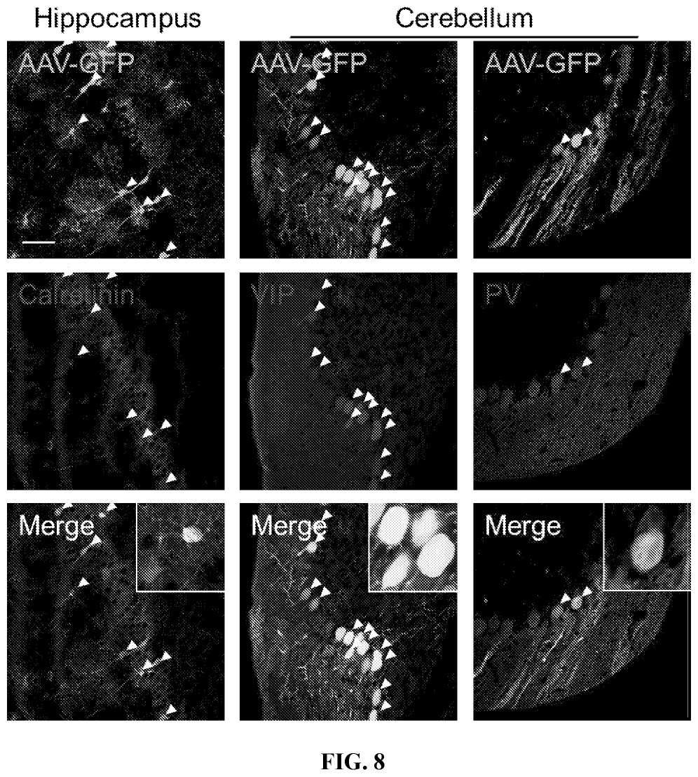

To further characterize the cell identities of transduced cells upon AAV-PHP.B IP injections, we performed immunostaining on cryopreserved brain sections. Selected antibodies against cellular markers of different interneuron subtypes were used. Notably, we found that at 14 d.p.i., a majority of AAV-transduced GFP-expressing cells (˜80%) in the hippocampus (CA1) are calretinin positive (FIG. 8). In the cerebellum, however, GFP-expressing cells were VIP+(˜60%) or PV+(˜40%).

### Example 5: Molecular and Behavioral Characterization of a Novel SSADHD Mouse Model, aldh5a1^{STOP/STOP}

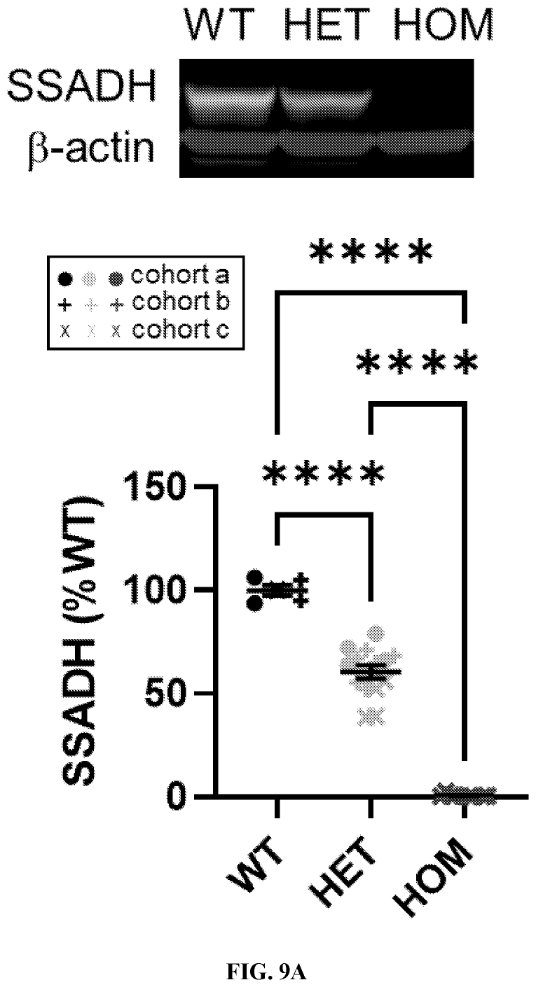

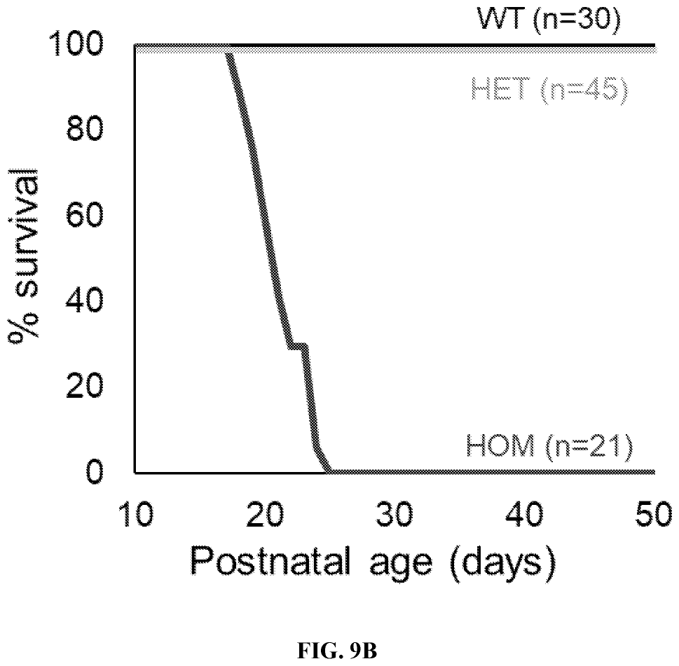

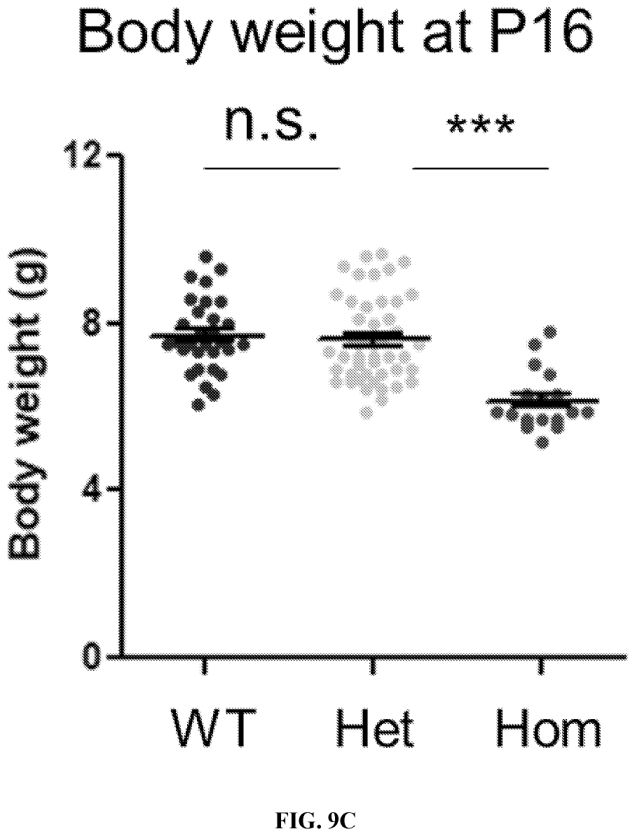

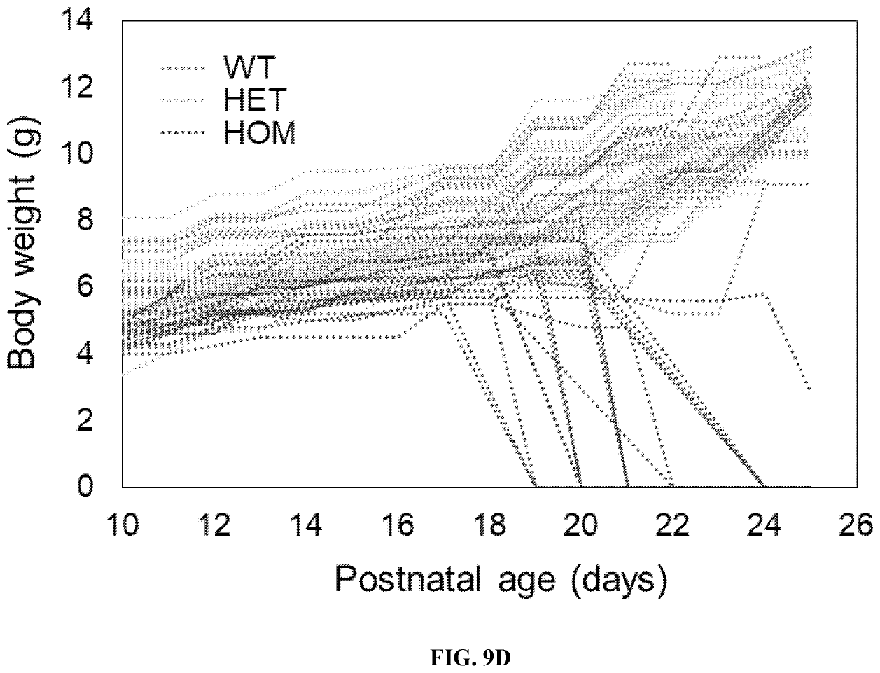

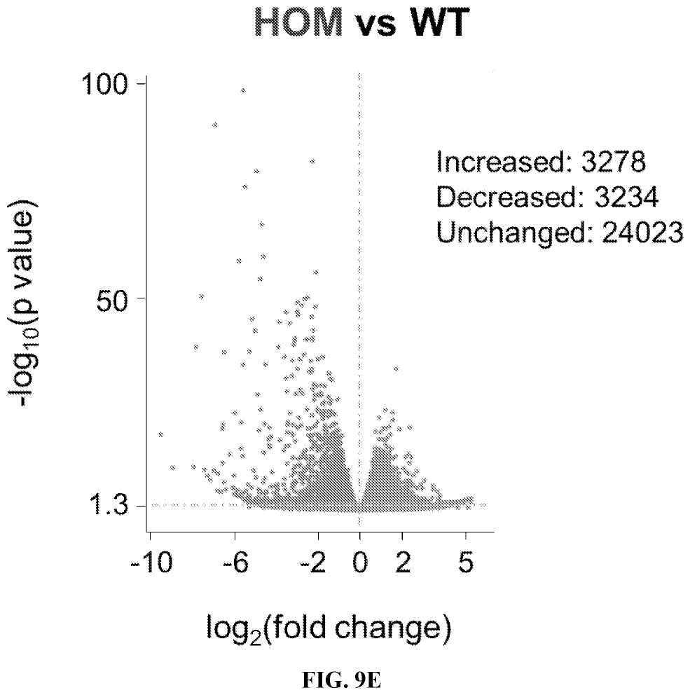

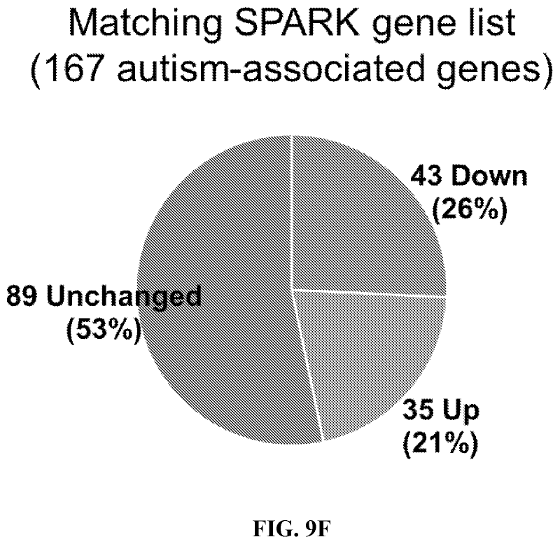

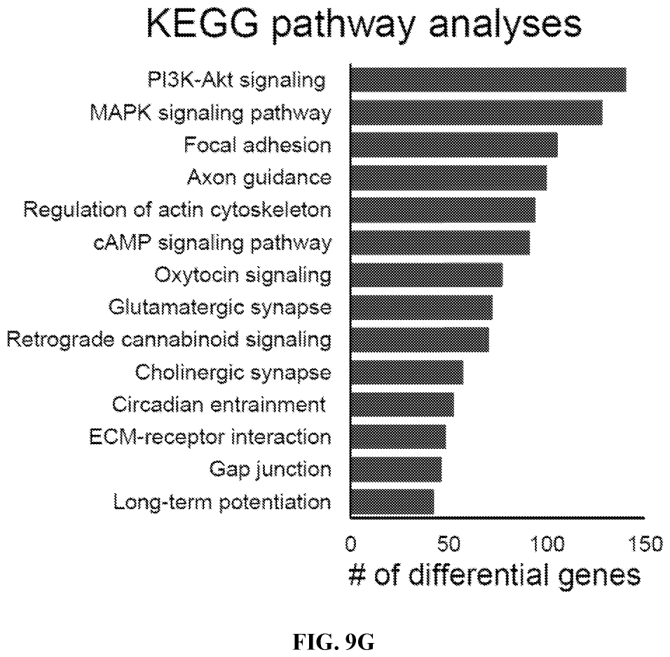

Molecular characterization of homozygous mutant HOM aldh5a1^{STOP/STOP} mice was performed by assessing SSADH expression and lethality. Homozygous mutant (HOM) aldh5a1^{STOP/STOP} mice did not express SSADH in the cortex (FIG. 9A). They also exhibited obligatory premature lethality before three weeks of postnatal age (FIG. 9B). Homozygous mutant (HOM) aldh5a1^{STOP/STOP} had lower body weight compared to wild-type (WT) and heterozygous mutant (TET) aldh5a1^{WT/STOP} mice (FIGS. 9C-9D). RNA-seq revealed thousands of differentially expressed genes between homozygous mutant (HOM) aldh5a1^{STOP/STOP} mice and wild-type (WT) mice (FIG. 9E). Many of the differentially expressed genes are associated with autism (FIG. 9F). KEGG pathway analysis revealed that the differentially expressed genes are associated with certain pathways (FIG. 9G).

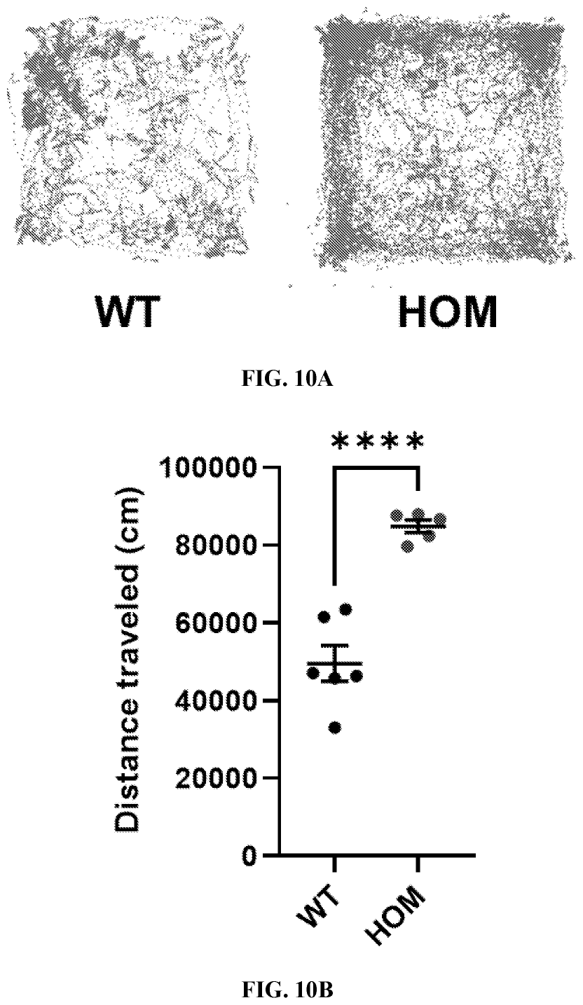

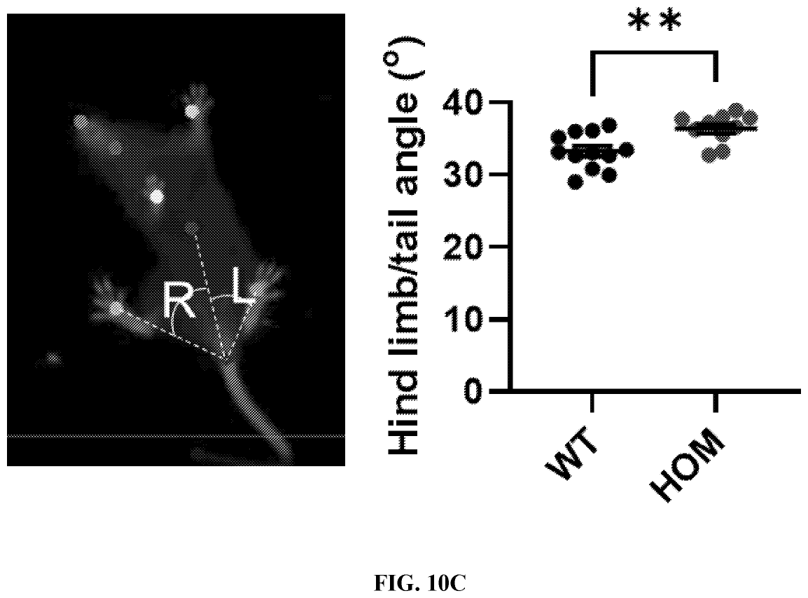

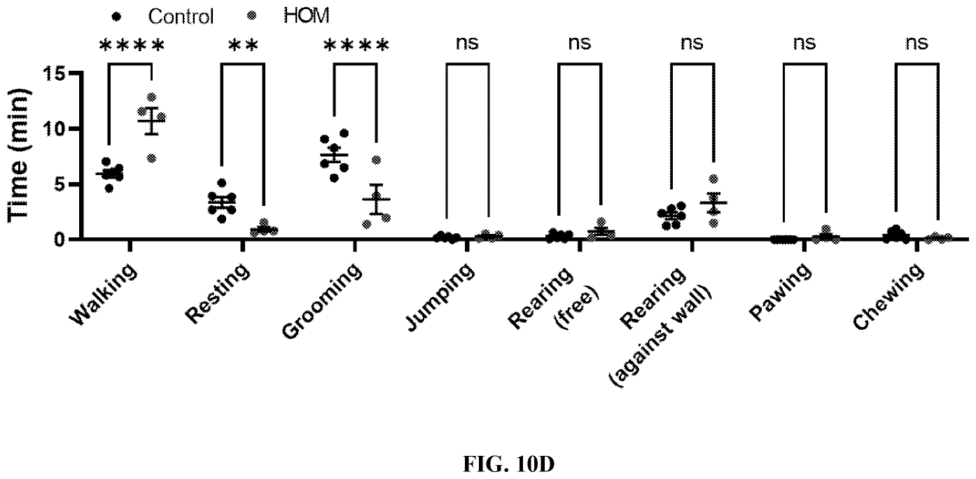

Behavioral characterization of homozygous mutant HOM aldh5a1^{STOP/STOP} mice was performed by assessing a variety of behaviors including walking, resting, grooming, jumping, rearing, pawing, and chewing. Homozygous mutant HOM aldh5a1^{STOP/STOP} mice exhibited hyperactivity compared to WT mice (FIGS. 10A-10B). Homozygous mutant HOM aldh5a1^{STOP/STOP} mice also displayed a gait abnormality compared to WT mice (FIG. 10C). Grooming and rearing behavior of homozygous mutant HOM aldh5a1^{STOP/STOP} mice also differed from that of WT mice (FIG. 10D).

Taken together, these results demonstrated that disruption of aldh5a1 gene leads to hyperactivity, gait abnormality, seizures, wide-range transcriptomic changes, somatic underdevelopment and premature lethality in mice.

### Example 6: Brain-Wide SSADH Restoration Leads to Appreciable Phenotypic Reversal and Enhanced Survival

This Example describes the impacts of brain-wide SSADH restoration using AAV-PHP.eB-Cre (referred to as AAV-Cre) to achieve brain-wide aldh5a1 restoration in aldh5a1^{lox-rtTA-STOP} mice.

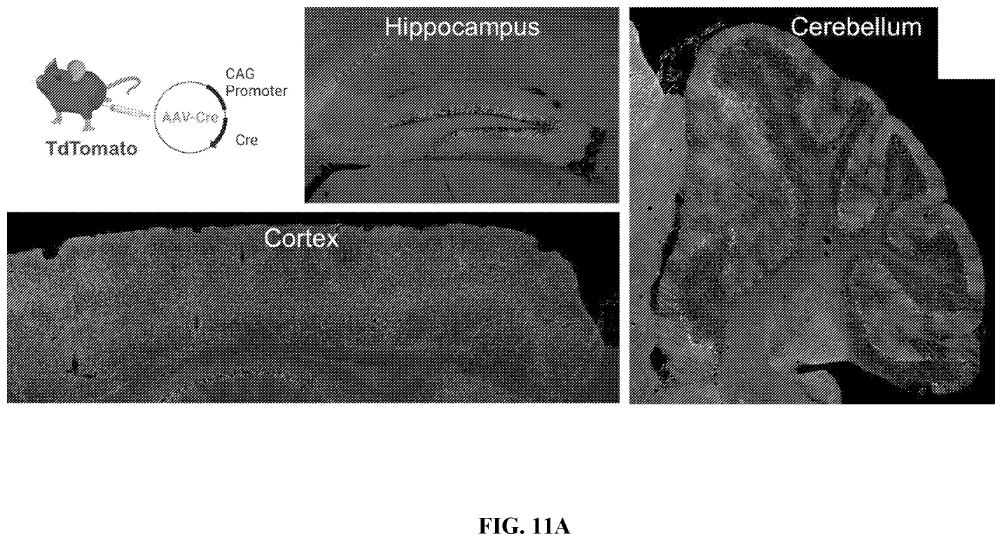

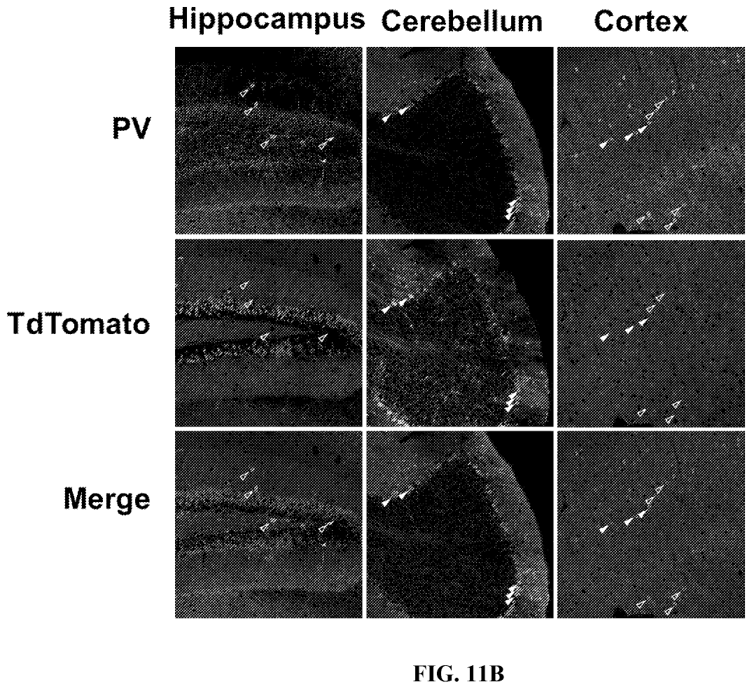

TdTomato mice were used to test the ability of AAV-Cre to mediate recombination. TdTomato mice are a Cre reporter tool strain designed to have a loxP-flanked STOP cassette preventing transcription of a CAG promoter-driven red fluorescent protein variant (tdTomato). TdTomato mice express robust tdTomato fluorescence following Cre-mediated recombination. TdTomato mice were injected with AAV-Cre and TdTomato expression was detected in the brain (FIG. 11A). TdTomato expression was also detected in PV+ cells (FIG. 11B).

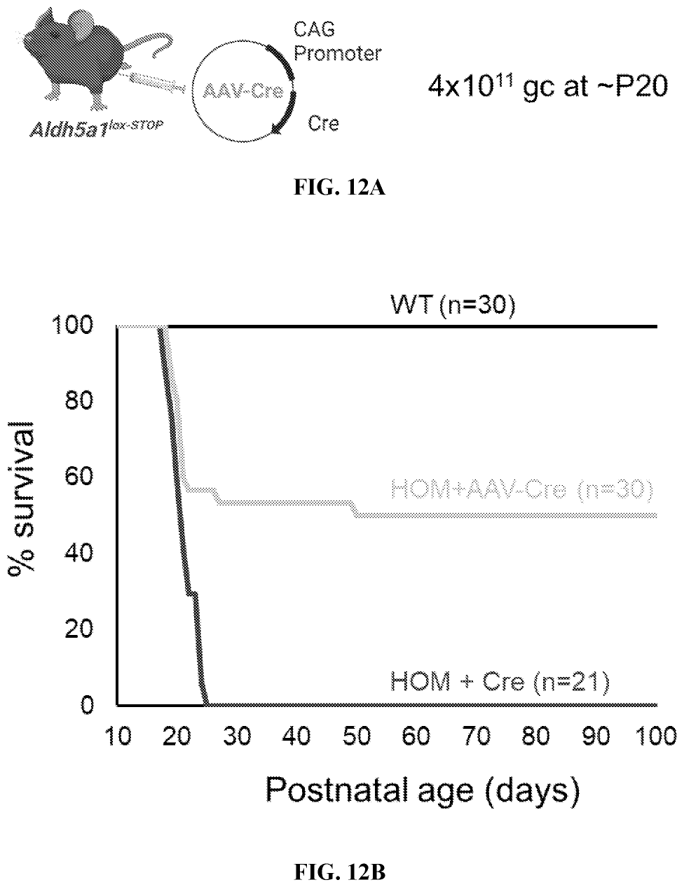

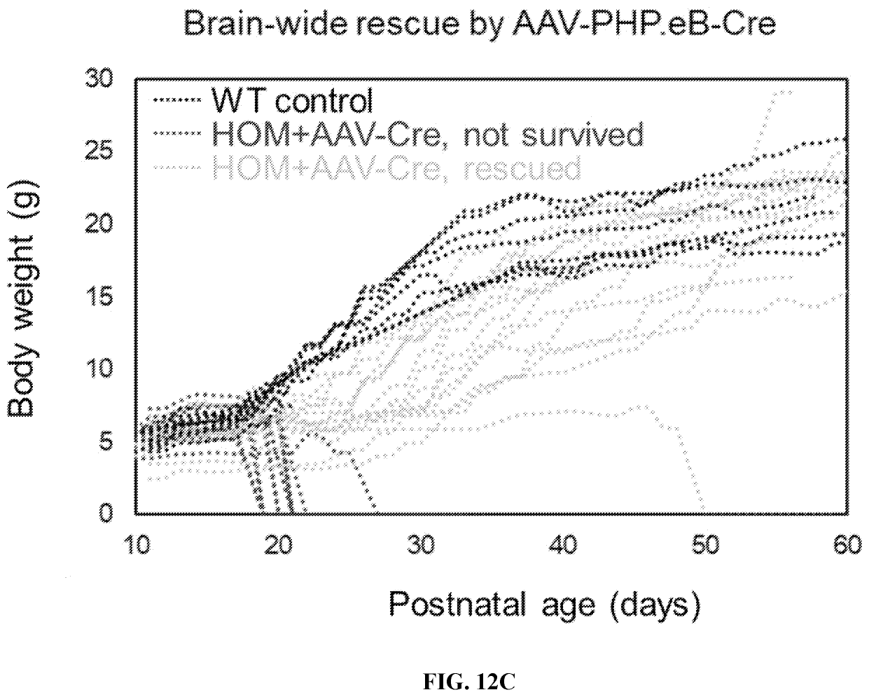

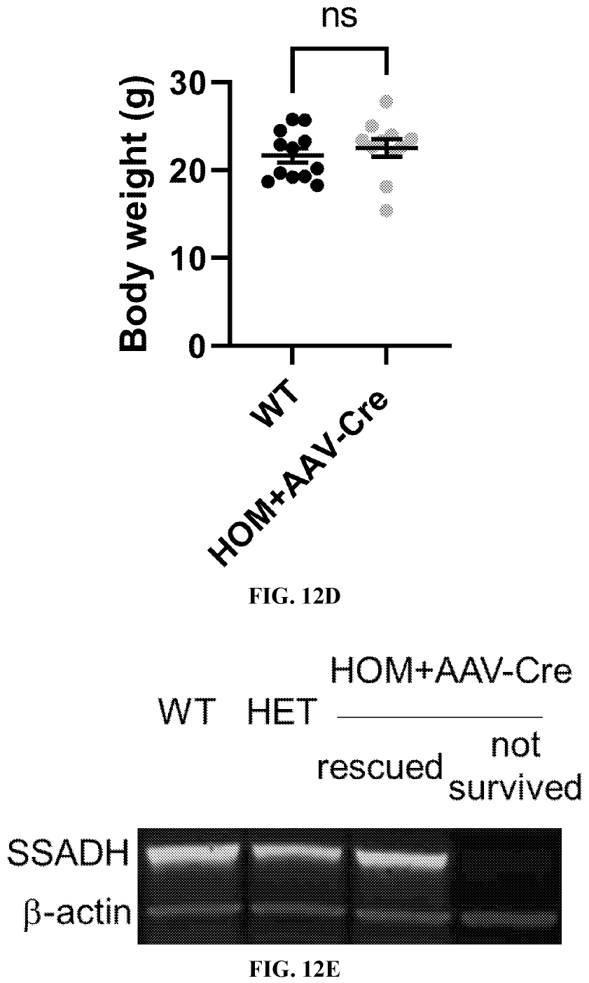

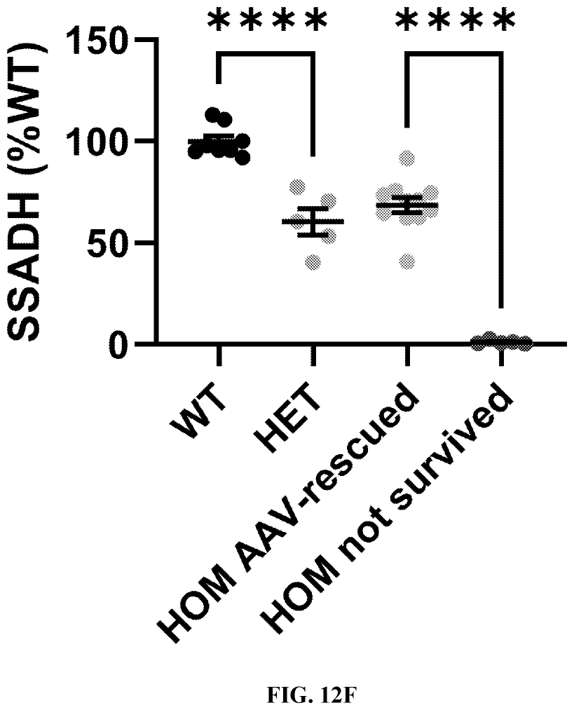

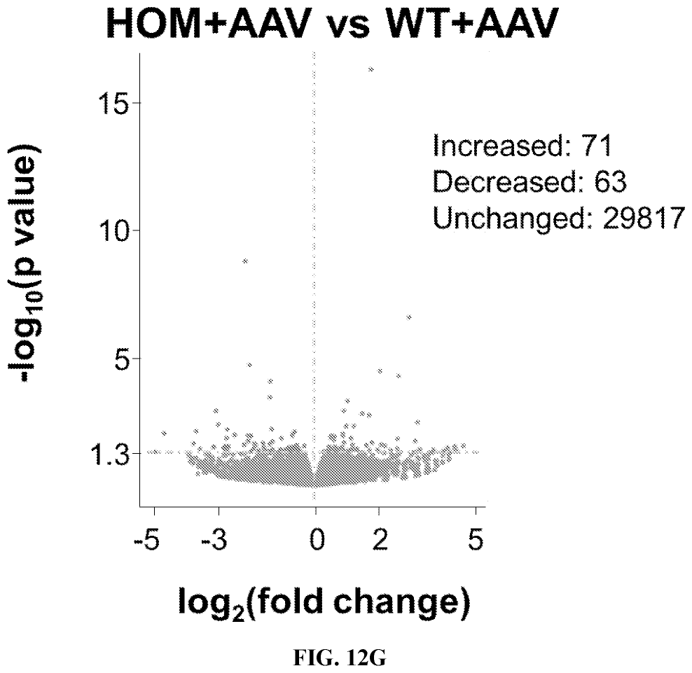

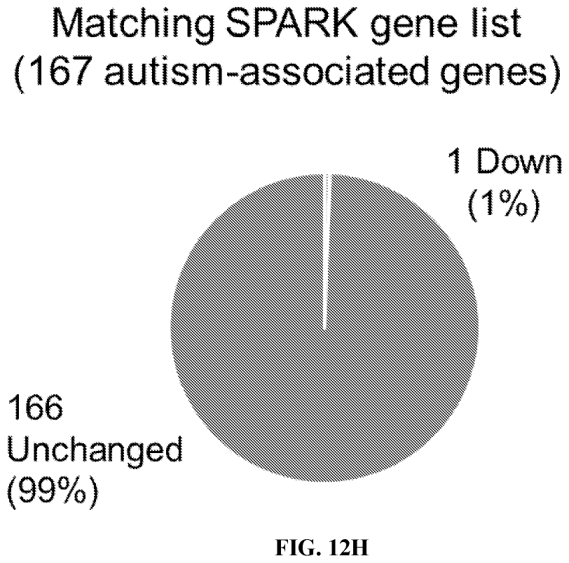

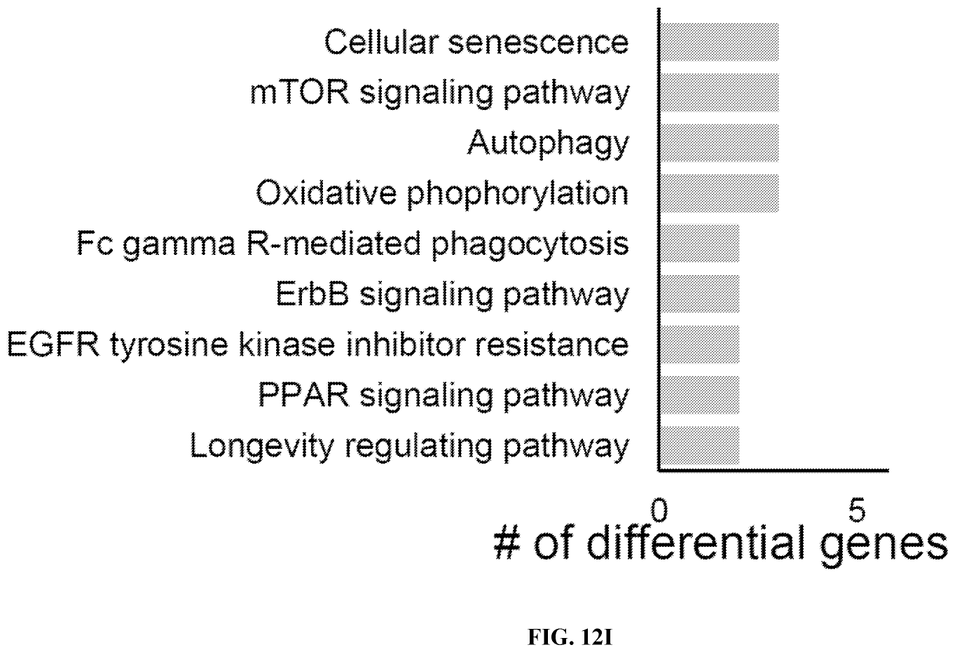

Brain-wide SSADH restoration was tested by injecting aldh5a1^{lox-rtTA-STOP} mice with AAV-Cre (4×10¹¹ genome copies) at postnatal age of 20 days (FIG. 12A). Molecular characterization of aldh5a1^{lox-rtTA-STOP} mice injected with AAV-Cre was performed. WT mice and aldh5a1^{lox-rtTA-STOP} mice without AAV-Cre injection were used as controls. Administration of AAV-Cre in aldh5a1^{lox-rtTA-STOP} mice rescued premature lethality (FIG. 12B) and low body weight observed in HOM mice (FIGS. 12C-12D). Mice administered AAV-Cre displayed increased brain-wide aldh5a1 restoration that correlated with survival (FIGS. 12E-12F) and rescued differentially expressed genes (FIGS. 12G-12H). KEGG pathway analysis revealed that the differentially expressed genes are associated with certain pathways (FIG. 12I).

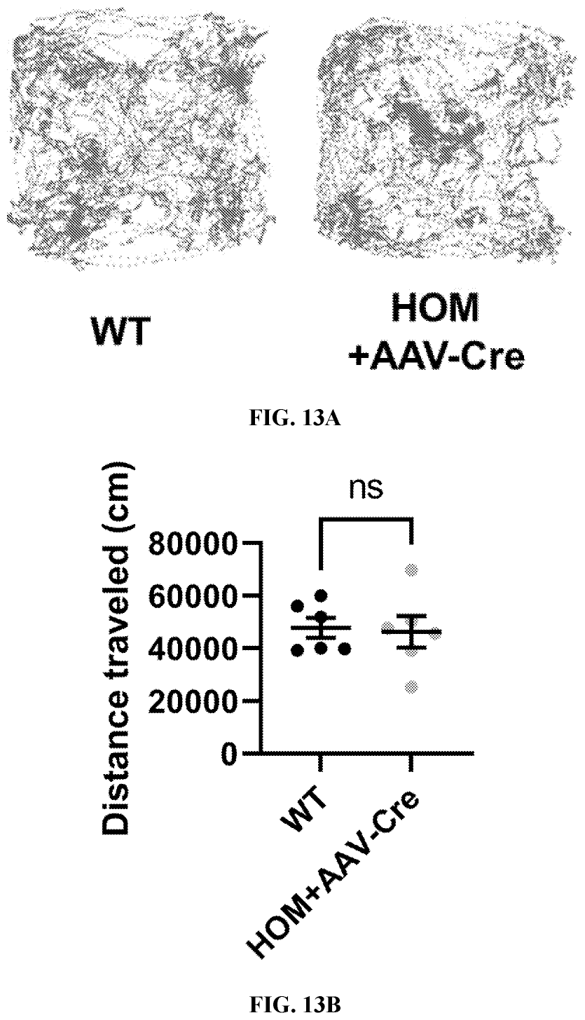

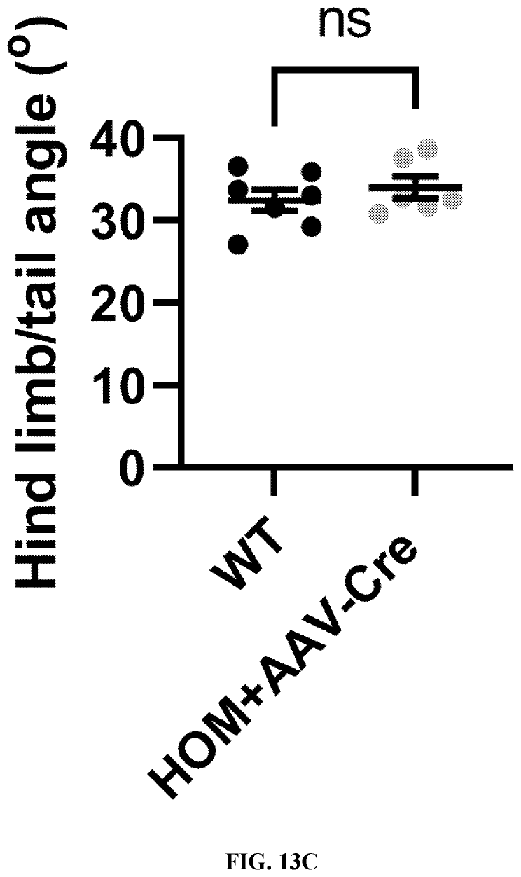

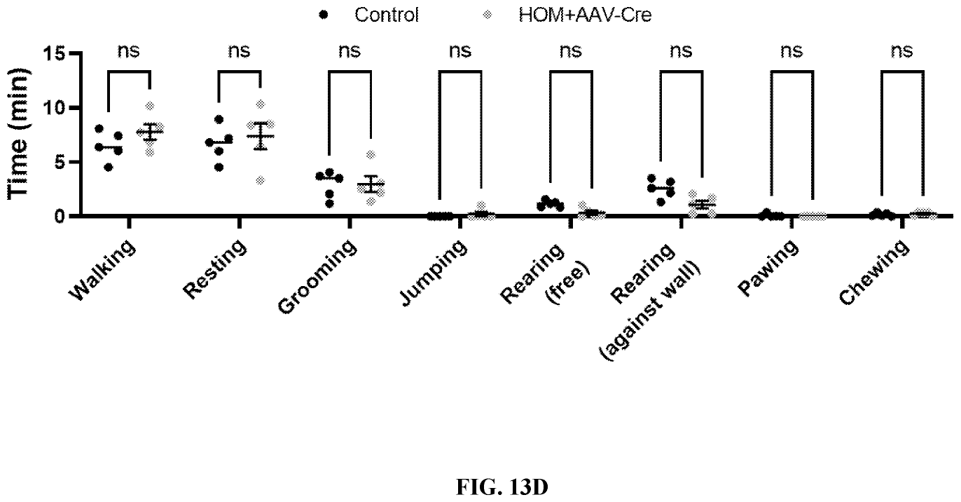

Behavioral characterization of homozygous mutant HOM aldh5a1^{STOP/STOP} mice administered AAV-Cre was performed by assessing a variety of behaviors including walking, resting, grooming, jumping, rearing, pawing, and chewing. After administration of AAV-Cre, aldh5a1^{lox-rtTA-STOP} mice exhibited behaviors similar to WT mice. For example, administration of AAV-Cre to aldh5a1^{lox-rtTA-STOP} mice rescued hyperactivity (FIGS. 13A-13B), gait abnormality (FIG. 13C), and grooming and rearing behaviors (FIG. 13D).

Taken together, these results demonstrate that single, large dose AAV-mediated brain-wide SSADH restoration at symptomatic stage leads to appreciable phenotypic reversal and enhanced survival.

### Example 7: PV+ Interneuron-Targeted SSADH Restoration Enhances Survival and Incompletely Rescues Pathological Phenotypes

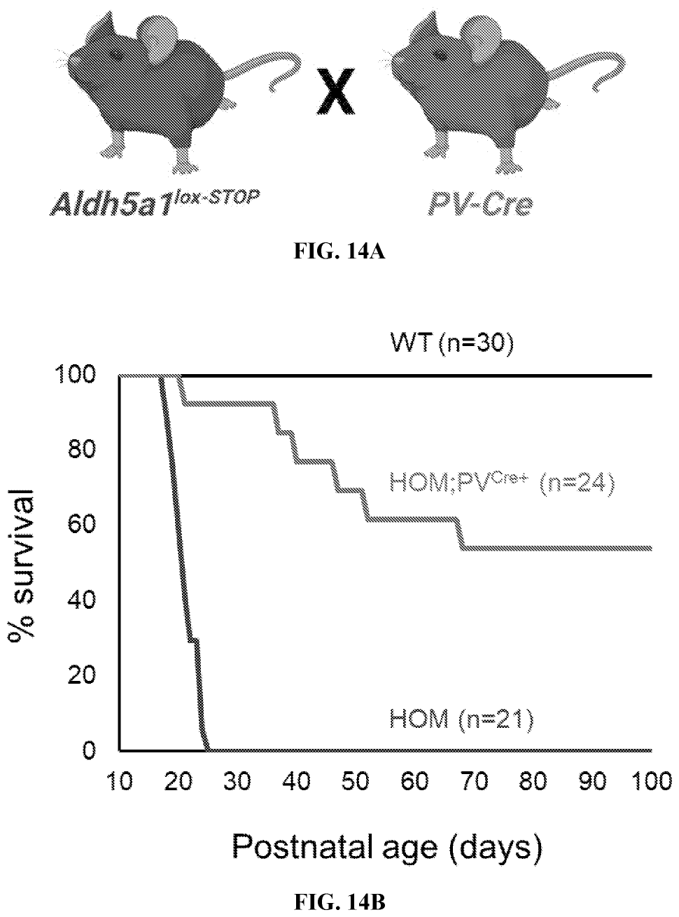

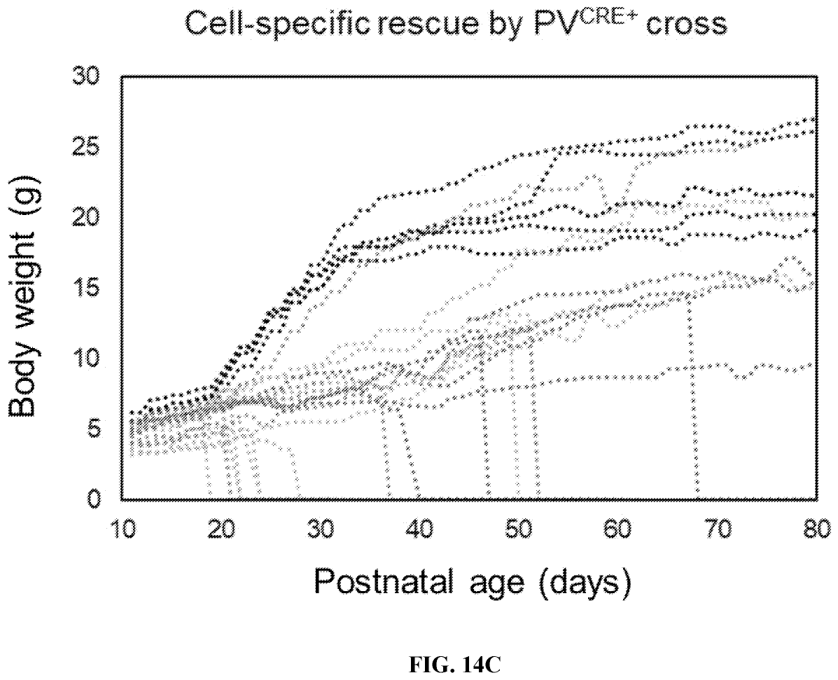

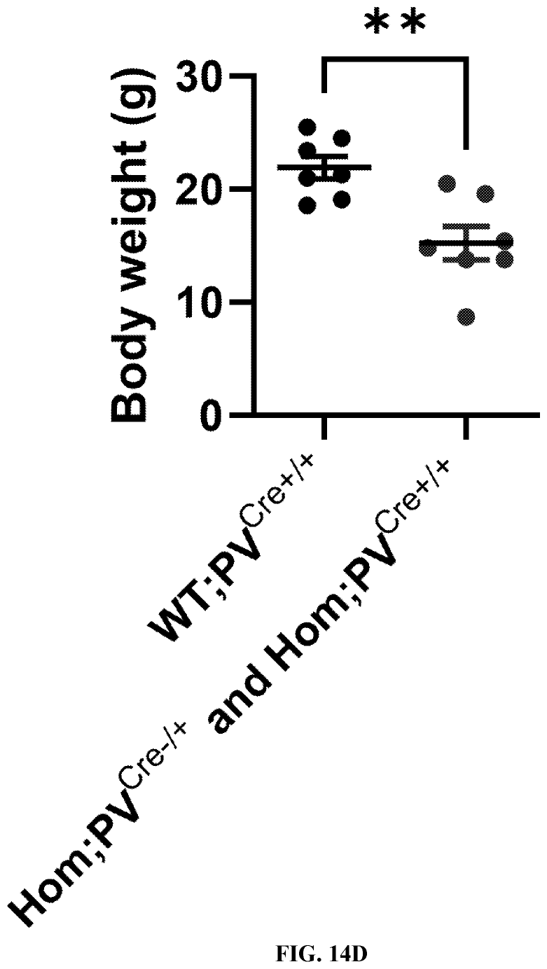

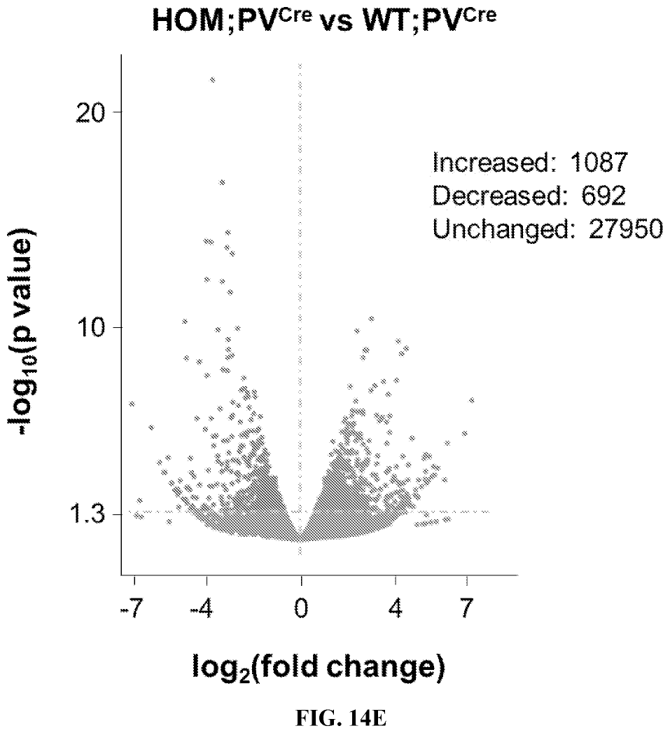

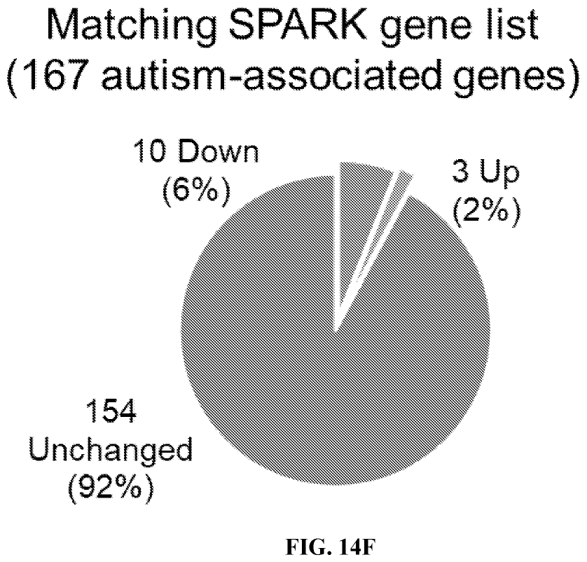

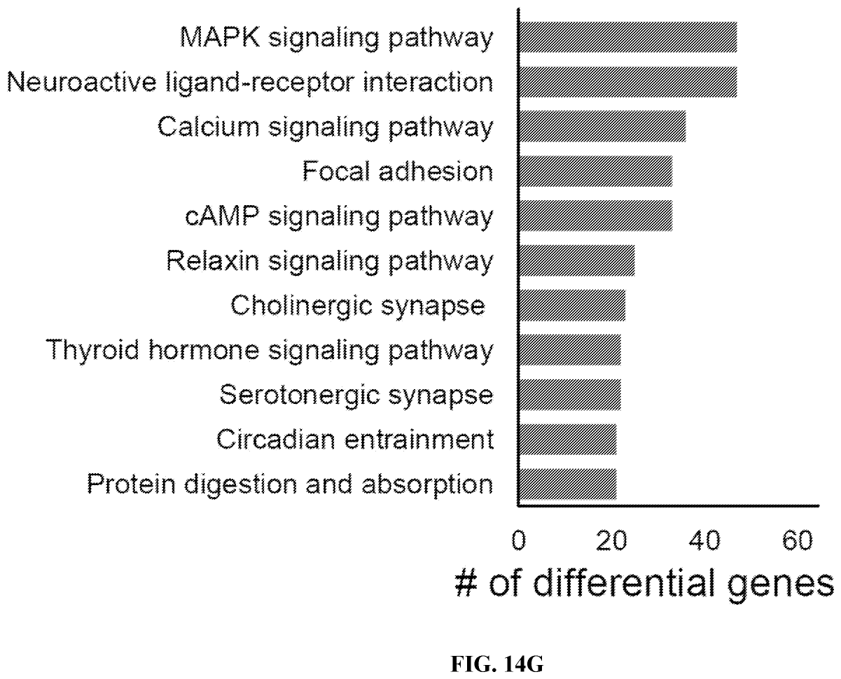

This Example describes the impacts of PV+ cell-specific SSADH restoration. HOM; PV^{Cre+} mice were produced by crossing aldh5a1^{lox-rtTA-STOP} mice and PV-Cre mice (FIG. 14A). Molecular characterization revealed partial rescue of premature lethality (FIG. 14B), low body weight (FIGS. 14C-14D), and differential gene expression (FIGS. 14E-14F). KEGG pathway analysis revealed that the differentially expressed genes are associated with certain pathways (FIG. 14G).

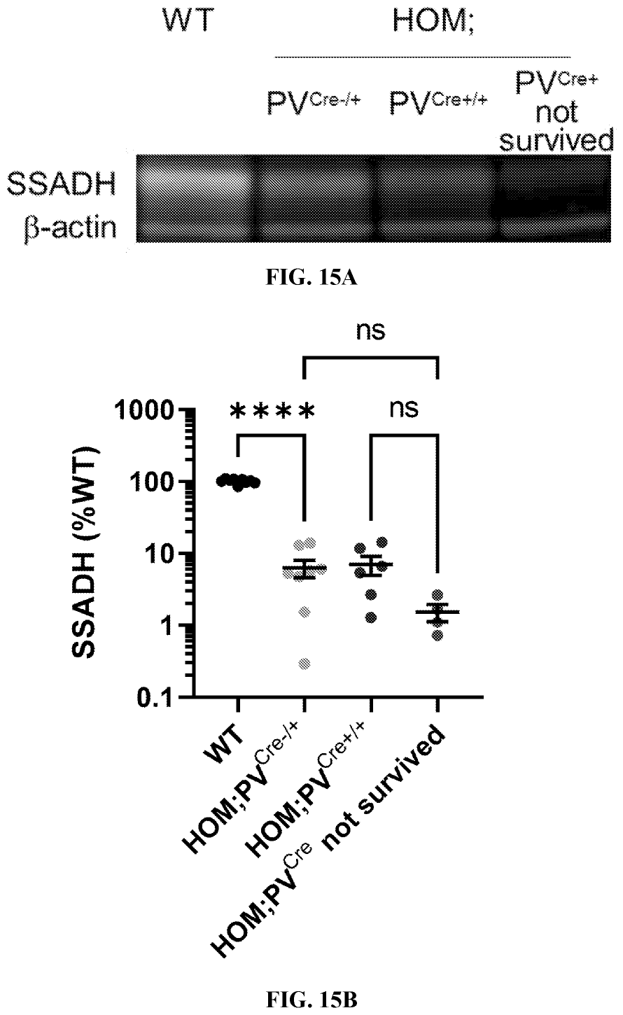

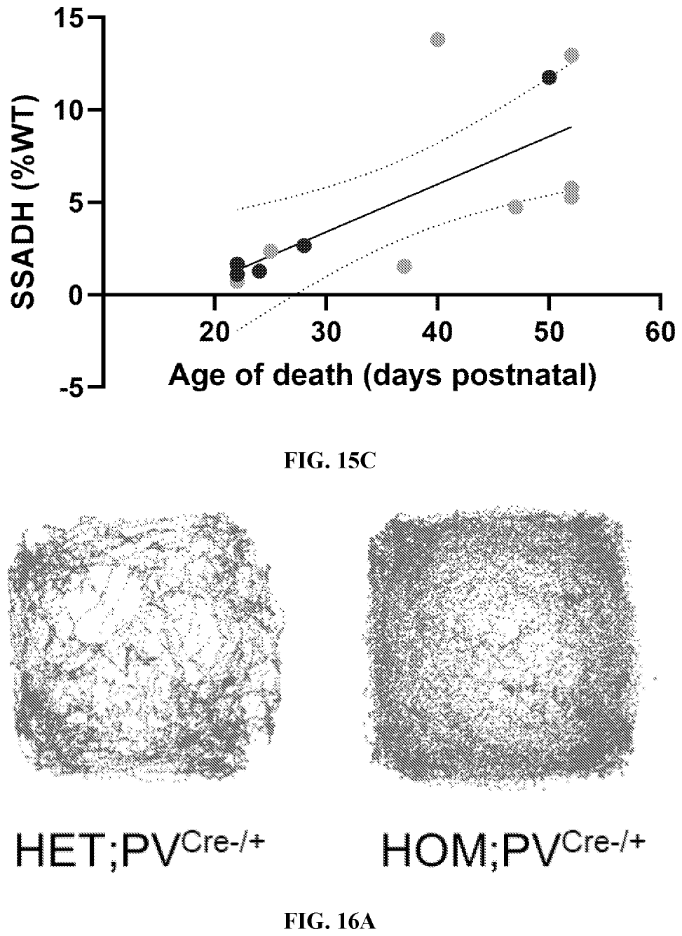

PV+ cell-specific SSADH restoration resulted in <15% of total SSADH protein restoration (FIGS. 15A-15B), yet this partial restoration was sufficient to enhance survival (FIG. 15C).

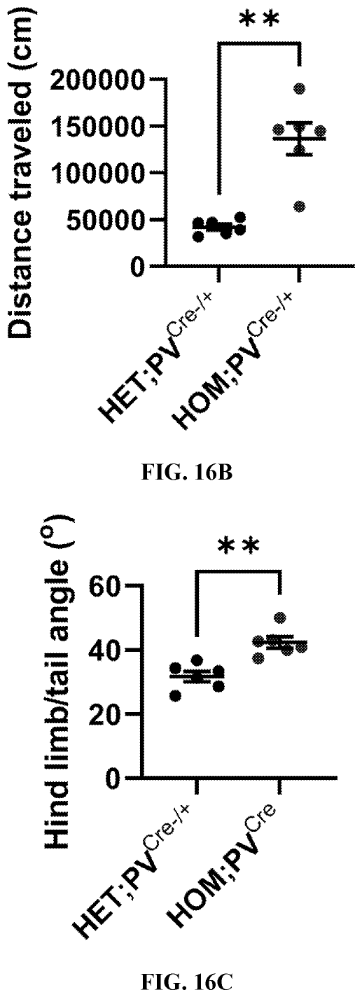

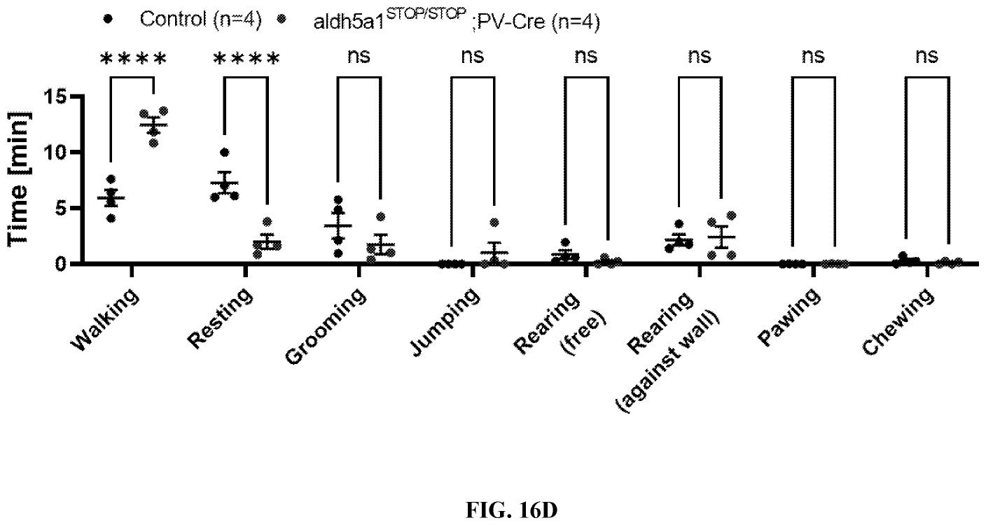

Behavioral characterization analysis showed incomplete behavior reversal upon PV-specific aldh5a1 restoration including incomplete reversal of hyperactivity (FIGS. 16A-16B), gait abnormality (FIG. 16C), and grooming and rearing behaviors (FIG. 16D).

Taken together, these results demonstrate that PV+ interneuron-targeted genetic rescue provided partial total SSADH protein restoration and enhanced survival, but residual pathological phenotypes persisted.

### Example 8: Brain-Wide Restoration Suppresses Seizures

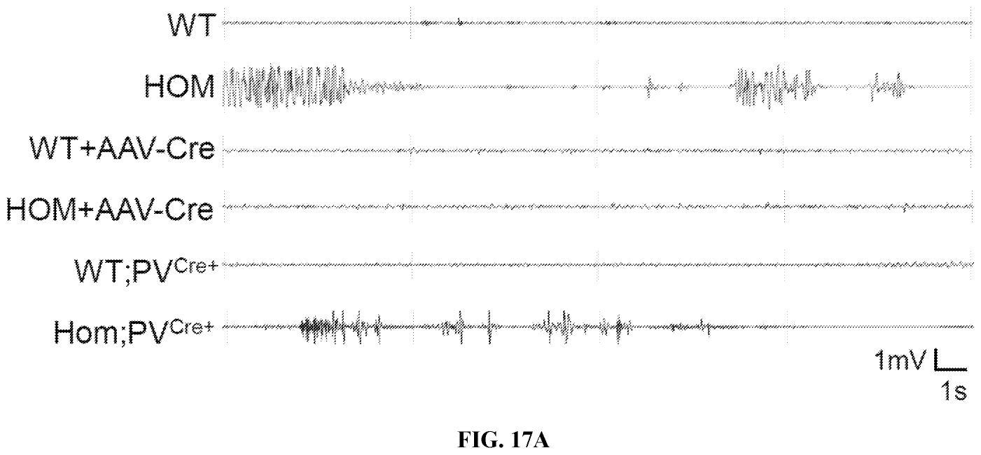

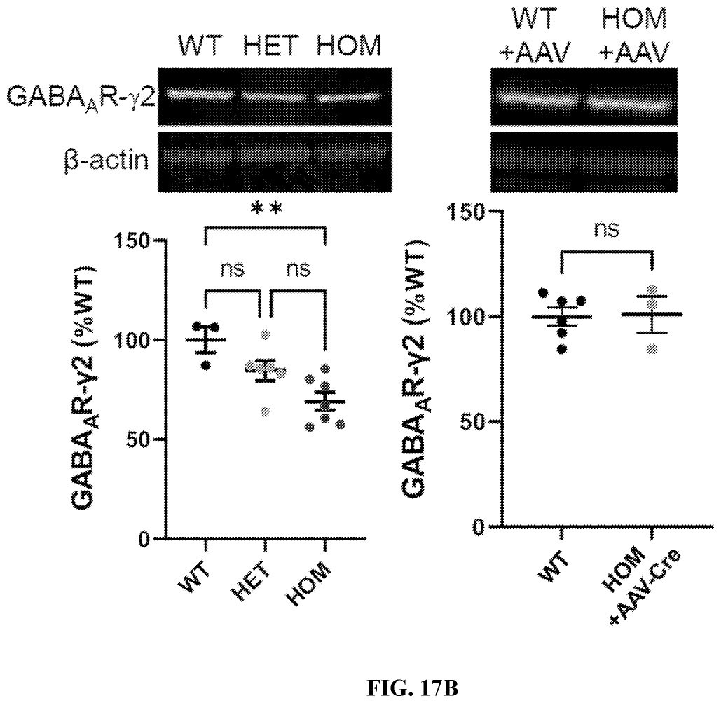

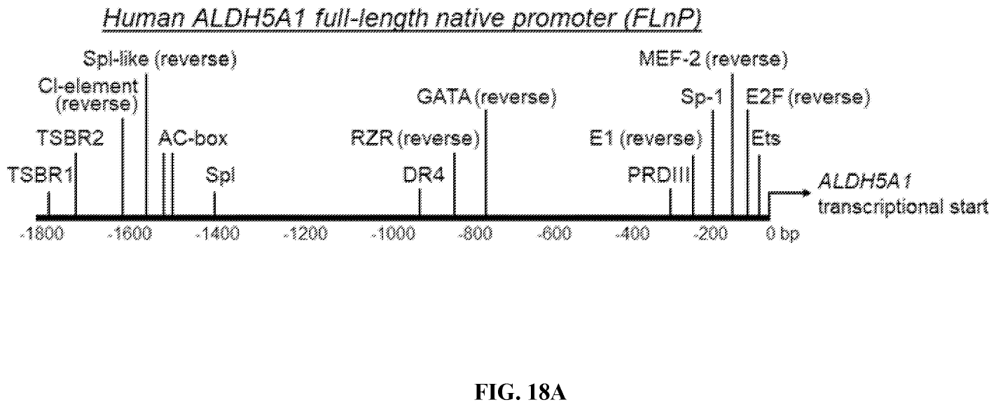

This Example describes the impact of brain-wide and PV-specific aldh5a1 restoration on seizure suppression. Brain-wide but not PV-specific aldh5a1 restoration suppressed seizures (FIG. 17A and Table 1). It was also shown that brain-wide aldh5a1 restoration restored GABA_{A}R-γ2 expression to WT levels (FIGS. 17B-17C).

TABLE 1
Results from seizure suppression studies

| | # of animals with generalized | | |
|---|---|---|---|
| | tonic-clonic seizure | | GTCS duration |
| | (GTCS) | | (s) |
| WT | 0/8 | (0%) | N/A |
| HET | 1/11 | (9%) | 4.30 |
| HOM | 4/5 | (80%) | 9.87 ± 2.69 |
| WT + AAV-Cre | 0/4 | (0%) | N/A |
| HOM + AAV-Cre | 0/5 | (0%) | N/A |
| WT; PV^{Cre+} | 0/5 | (0%) | N/A |
| HOM; PV^{Cre+} | 4/8 | (50%) | 7.57 ± 4.51 |

### Example 9: Vector Design for Gene Therapy in SSADHD

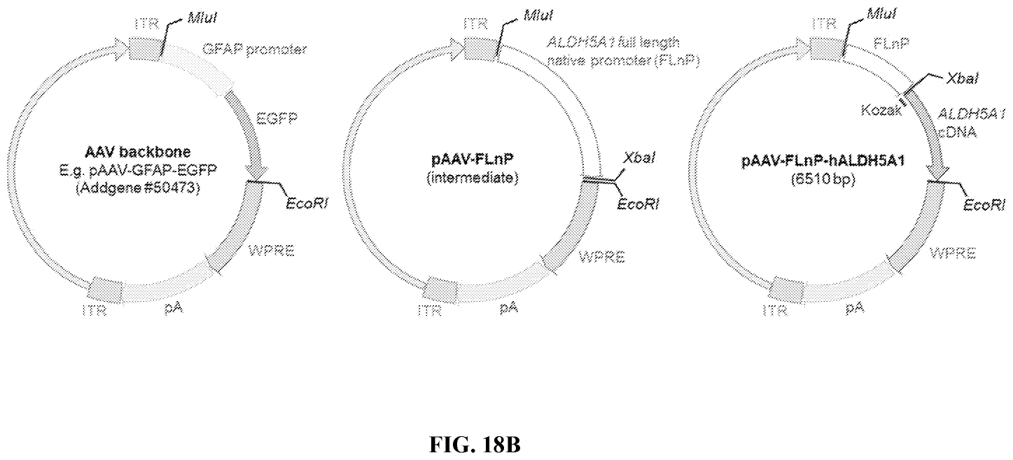

This Example describes the use of full-length native promoter (FLnP) of ALDH5A1 to drive SSADH functional expression using a gene therapy vector (e.g., AAV) vector (FIG. 18A). The genomic sequence upstream of the ALDH5A1 transcriptional start site harboring regulatory motifs is specially designed to drive SSADH expression mimicking its endogenous profile in terms of cell type specificity and level of expression. This specific vector is designed to allow highly regulated ALDH5A1 expression without overexpression, which can be oncogenic.

Human ALDH5A1 promoter sequence was previously published (Blasi P et al., Mol Genet Metab 2002; 76:348-362). We further defined this 1.8 kb region in the human genomic sequence directly upstream of the ALDH5A1 transcriptional start site. This genomic region, found in human chromosome 6, is analyzed by sequence motif database, revealing the presence of multiple transcriptional regulatory sites. This includes a cluster of transcriptional regulatory motifs 0.3 kb proximal to the transcriptional start, as well as another cluster at 1.4-1.8 kb region further upstream. This totality of 1.8 kb region, defined as the FLnP, is subcloned into an AAV vector together with the recombinant cDNA of SSADH in a two-step cloning strategy (FIG. 18B). This tandem sequence of FLnP-ALDH5A1 (SEQ ID NO:3) is a unique sequence we specifically design for this AAV vector, which is not found in nature nor previously unavailable. Primer sequences are listed in figure therein.

For Cloning pAAV-FLnP:

### Mlul-FLnP-Xba1-EcoRI Fragment (833 bp)

FLnP-F:
(SEQ ID NO: 4)
AAACGCGTCTAGTATGACTTTGAACGCTATATTGAATTTAATGTGGC
FLnP-R:
(SEQ ID NO: 5)
AAGAATTCAATCTAGACAACGACGGCGACAGGAAACAGG

For Cloning pAAV-FLnP-hALDH5A1:

### Xba1-Kozak-ALDH5A1-EcoRI (1665 bp)

ALDH5A1-F:
(SEQ ID NO: 6)
AATCTAGAGCCACCATGGCGACCTGCATTTGGCTG
ALDH5A1-R:
(SEQ ID NO: 7)
AAGAATTCCTACAAGCCCCCGTAACACACA

### REFERENCES

1. Gibson K M et al. Succinic semialdehyde dehydrogenase deficiency: an inborn error of gamma-aminobutyric acid metabolism. Clin Chim Acta 133, 33-42, (1983).
2. Pearl P L et al. Inherited disorders of gamma-aminobutyric acid metabolism and advances in ALDH5A1 mutation identification. Dev Med Child Neurol 57, 611-617, (2015).
3. Jakobs C et al. Urinary excretion of gamma-hydroxybutyric acid in a patient with neurological abnormalities. The probability of a new inborn error of metabolism. Clin Chim Acta 111, 169-178, (81)90184-4 (1981).
4. Pearl P L, Wiwattanadittakul N, Roullet J B & Gibson K M in GeneReviews((R)) (eds Adam M P et al.) (1993).
5. Malaspina P et al. Succinic semialdehyde dehydrogenase deficiency (SSADHD): Pathophysiological complexity and multifactorial trait associations in a rare monogenic disorder of GABA metabolism. Neurochem Int 99, 72-84, (2016).
6. Jansen E E et al. Correlation of blood biomarkers with age informs pathomechanisms in succinic semialdehyde dehydrogenase deficiency (SSADHD), a disorder of GABA metabolism. J Inherit Metab Dis 39, 795-800, (2016).
7. Pearl P L, Shukla L, Theodore W H, Jakobs C & Michael Gibson K Epilepsy in succinic semialdehyde dehydrogenase deficiency, a disorder of GABA metabolism. Brain Dev 33, 796-805, (2011).
8. Pearl P L et al. Decreased GABA-A binding on FMZ-PET in succinic semialdehyde dehydrogenase deficiency. Neurology 73, 423-429, (2009).
9. Parviz M, Vogel K, Gibson K M & Pearl P L Disorders of GABA metabolism: SSADH and GABA-transaminase deficiencies. J Pediatr Epilepsy 3, 217-227, (2014).
10. Pearl P L et al. Taurine trial in succinic semialdehyde dehydrogenase deficiency and elevated CNS GABA. Neurology 82, 940-944, (2014).
11. Pearl P L et al. Succinic semialdehyde dehydrogenase deficiency: lessons from mice and men. J Inherit Metab Dis 32, 343-352, (2009).
12. Vogel K R et al. Thirty years beyond discovery-clinical trials in succinic semialdehyde dehydrogenase deficiency, a disorder of GABA metabolism. J Inherit Metab Dis 36, 401-410, (2013).
13. Gropman A Vigabatrin and newer interventions in succinic semialdehyde dehydrogenase deficiency. Ann Neurol 54 Suppl 6, S66-72, (2003).
14. Vogel K R et al. Succinic semialdehyde dehydrogenase deficiency, a disorder of GABA metabolism: an update on pharmacological and enzyme-replacement therapeutic strategies. J Inherit Metab Dis 41, 699-708, (2018).
15. Gupta M et al. Liver-directed adenoviral gene transfer in murine succinate semialdehyde dehydrogenase deficiency. Mol Ther 9, 527-539, (2004).
16. Ganguly K, Schinder A F, Wong S T & Poo M GABA itself promotes the developmental switch of neuronal GABAergic responses from excitation to inhibition. Cell 105, 521-532, (2001).
17. Rivera C et al. The K+/Cl− co-transporter KCC2 renders GABA hyperpolarizing during neuronal maturation. Nature 397, 251-255, (1999).
18. Lee H H, Deeb T Z, Walker J A, Davies P A & Moss S J NMDA receptor activity downregulates KCC2 resulting in depolarizing GABA_{A} receptor-mediated currents. Nat Neurosci 14, 736-743, (2011).
19. Jacob T C, Moss S J & Jurd R GABA(A) receptor trafficking and its role in the dynamic modulation of neuronal inhibition. Nat Rev Neurosci 9, 331-343, (2008).
20. Luscher B, Fuchs T & Kilpatrick C L GABA_{A} receptor trafficking-mediated plasticity of inhibitory synapses. Neuron 70, 385-409, (2011).
21. Hensch T K Critical period plasticity in local cortical circuits. Nat Rev Neurosci 6, 877-888, (2005).
22. Reh R K et al. Critical period regulation across multiple timescales. Proc Natl Acad Sci USA 117, 23242-23251, (2020).
23. Huang Z J et al. BDNF regulates the maturation of inhibition and the critical period of plasticity in mouse visual cortex. Cell 98, 739-755, (1999).
24. Lee H H C et al. Genetic Otx2 mis-localization delays critical period plasticity across brain regions. Mol Psychiatry 22, 680-688, (2017).
25. Miyata S, Komatsu Y, Yoshimura Y, Taya C & Kitagawa H Persistent cortical plasticity by upregulation of chondroitin 6-sulfation. Nat Neurosci 15, 414-422, 5411-412, (2012).
26. Gervain J et al. Valproate reopens critical-period learning of absolute pitch. Front Syst Neurosci 7, 102, (2013).
27. Hogema B M et al. Pharmacologic rescue of lethal seizures in mice deficient in succinate semialdehyde dehydrogenase. Nat Genet 29, 212-216, (2001).
28. Desai A K, Li C, Rosenberg A S & Kishnani P S Immunological challenges and approaches to immunomodulation in Pompe disease: a literature review. Ann Transl Med 7, 285, (2019).
29. Shirley J L, de Jong Y P, Terhorst C & Herzog R W Immune Responses to Viral Gene Therapy Vectors. Mol Ther 28, 709-722, (2020).
30. Le Magueresse C & Monyer H GABAergic interneurons shape the functional maturation of the cortex. Neuron 77, 388-405, (2013).
31. Blasi P et al. Structure of human succinic semialdehyde dehydrogenase gene: identification of promoter region and alternatively processed isoforms. Mol Genet Metab 76, 348-362, (2002).
32. Zeisel A et al. Brain structure. Cell types in the mouse cortex and hippocampus revealed by single-cell RNA-seq. Science 347, 1138-1142, (2015).
33. Hsieh T H et al. Trajectory of Parvalbumin Cell Impairment and Loss of Cortical Inhibition in Traumatic Brain Injury. Cereb Cortex 27, 5509-5524, (2017).
34. Paxinos G & Franklin K B J Paxinos and Franklin's the mouse brain in stereotaxic coordinates. 4th edn, (Elsevier/AP, 2013).
35. Devito L M, Kanter B R & Eichenbaum H The hippocampus contributes to memory expression during transitive inference in mice. Hippocampus 20, 208-217, (2010).
36. Kaemmerer W F et al. In vivo transduction of cerebellar Purkinje cells using adeno-associated virus vectors. Mol Ther 2, 446-457, (2000).
37. Quadros R M et al. Easi-CRISPR: a robust method for one-step generation of mice carrying conditional and insertion alleles using long ssDNA donors and CRISPR ribonucleoproteins. Genome Biol 18, 92, (2017).
38. Coolidge C J, Seely R J & Patton J G Functional analysis of the polypyrimidine tract in pre-mRNA splicing. Nucleic Acids Res 25, 888-896, (1997).
39. Taniguchi H et al. A resource of Cre driver lines for genetic targeting of GABAergic neurons in cerebral cortex. Neuron 71, 995-1013, (2011).
40. Concolino D, Deodato F & Parini R Enzyme replacement therapy: efficacy and limitations. Ital J Pediatr 44, 120, (2018).
41. Toscano M G et al. Physiological and tissue-specific vectors for treatment of inherited diseases. Gene Ther 18, 117-127, (2011).
42. Didiasova M et al. Succinic Semialdehyde Dehydrogenase Deficiency: An Update. Cells 9, (2020).
43. Zeng H et al. An inducible and reversible mouse genetic rescue system. PLoS Genet 4, e1000069, (2008).
44. Akaboshi S et al. Mutational spectrum of the succinate semialdehyde dehydrogenase (ALDH5A1) gene and functional analysis of 27 novel disease-causing mutations in patients with SSADH deficiency. Hum Mutat 22, 442-450, (2003).
45. Akiyama T et al. SSADH deficiency possibly associated with enzyme activity-reducing SNPs. Brain Dev 38, 871-874, (2016).
46. Collard R, Majtan T, Park I & Kraus J P Import of TAT-Conjugated Propionyl Coenzyme A Carboxylase Using Models of Propionic Acidemia. Mol Cell Biol 38, (2018).
47. Lam D et al. Tissue-specific extracellular matrix accelerates the formation of neural networks and communities in a neuron-glia co-culture on a multi-electrode array. Sci Rep 9, 4159, (2019).
48. Beurdeley M et al. Otx2 binding to perineuronal nets persistently regulates plasticity in the mature visual cortex. J Neurosci 32, 9429-9437, (2012).
49. Faedo A et al. Differentiation of human telencephalic progenitor cells into MSNs by inducible expression of Gsx2 and Ebfl. Proc Natl Acad Sci USA 114, E1234-E1242, (2017).
50. Loew R, Heinz N, Hampf M, Bujard H & Gossen M Improved Tet-responsive promoters with minimized background expression. BMC Biotechnol 10, 81, (2010).
51. Lapalme-Remis S et al. Natural history of succinic semialdehyde dehydrogenase deficiency through adulthood. Neurology 85, 861-865, (2015).
52. Deverman B E et al. Cre-dependent selection yields AAV variants for widespread gene transfer to the adult brain. Nat Biotechnol 34, 204-209, (2016).
53. Hordeaux J et al. The Neurotropic Properties of AAV-PHP.B Are Limited to C57BL/6J Mice. Mol Ther 26, 664-668, (2018).
54. Bei F et al. Restoration of Visual Function by Enhancing Conduction in Regenerated Axons. Cell 164, 219-232, (2016).
55. Hordeaux J et al. The GPI-Linked Protein L Y6A Drives AAV-PHP.B Transport across the Blood-Brain Barrier. Mol Ther 27, 912-921, (2019).
56. Huang Q et al. Delivering genes across the blood-brain barrier: LY6A, a novel cellular receptor for AAV-PHP.B capsids. PLoS One 14, e0225206, (2019).
57. Matsuzaki Y et al. Intravenous administration of the adeno-associated virus-PHP.B capsid fails to upregulate transduction efficiency in the marmoset brain. Neurosci Lett 665, 182-188, (2018).
58. Naso M F, Tomkowicz B, Perry W L 3rd & Strohl W R Adeno-Associated Virus (AAV) as a Vector for Gene Therapy. BioDrugs 31, 317-334, (2017).
59. Lundstrom K, Viral Vectors in Gene Therapy. Diseases 6, (2018).
60. Liu Y, Hegarty S, Winter C, Wang F & He Z Viral vectors for neuronal cell type-specific visualization and manipulations. Curr Opin Neurobiol 63, 67-76, (2020).
61. High-dose AAV gene therapy deaths. Nat Biotechnol 38, 910, (2020).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating succinic semialdehyde dehydrogenase deficiency (SSADHD) in a subject, the method comprising administering to the subject an effective amount of a polynucleotide comprising a promoter sequence and a sequence encoding succinic semialdehyde dehydrogenase (SSADH).

2. The method of claim 1, wherein the promoter sequence is a neuron-specific promoter sequence.

3. The method of claim 1, wherein the promoter sequence is a γ-aminobutyric acid (GABA) transporter 1 (GAT1) promoter sequence, a GABA transporter 3 (GAT1) promoter sequence, a 5-hydroxytryptamin receptor (5HT3R) promoter sequence, a somatostatin (SST) promoter sequence, a parvalbumin (PV) promoter sequence, a Ca²⁺/calmodulin-dependent kinase subunit α (CaMKII) promoter sequence, neuron-specific enolase (NSE) promoter sequence, synapsin I with a minimal CMV sequence (Syn I-minCMV) promoter sequence, or a aldehyde dehydrogenase 5 family member A1 (ALDH5A1) promoter sequence.

4. The method of claim 1, wherein the promoter sequence comprises a nucleotide sequence having at least 90%, at least 95%, at least 98%, at least 99% or 100% identity to SEQ ID NO:2.

5. The method of claim 1, wherein the sequence encoding SSADH comprises a nucleotide sequence having at least 90%, at least 95%, at least 98%, at least 99% or 100% identity to SEQ ID NO:1.

6. The method of claim 1, wherein the polynucleotide comprises a nucleotide sequence having at least 90%, at least 95%, at least 98%, at least 99% or 100% identity to SEQ ID NO:3.

7. The method of claim 1, wherein the polynucleotide is administered to the central nervous system (CNS) of the subject.

8. The method of claim 1, wherein the administering of the polynucleotide results in expression of SSADH in the brain of the subject.

9. The method of claim 1, wherein the administering of the polynucleotide results in expression of SSADH in parvalbumin-positive interneurons of the subject.

10. The method of claim 1, wherein the subject is a human.

11. The method of claim 1, wherein the polynucleotide is a viral vector.

12. The method of claim 11, wherein the viral vector is a lentivirus vector, an alphavirus vector, an enterovirus vector, a pestivirus vector, a baculovirus vector, a herpesvirus vector, an Epstein Barr virus vector, a papovavirus vector, a poxvirus vector, a vaccinia virus vector, herpes simplex virus vector, an adenovirus vector, or an adeno-associated virus (AAV) vector.

13. The method of claim 12, wherein the (AAV) vector is packaged in an AAV particle.

14. The method of claim 13, wherein the AAV particle comprises capsid proteins derived from AAV9 serotype.

15. The method of claim 13, wherein the AAV particle comprises a capsid protein variant derived from AAV9 serotype.

16. The method of claim 15, wherein the capsid protein variant derived from AAV9 comprises PHP.B capsid or PHP.eB capsid.

17. A polynucleotide comprising a nucleotide sequence having at least 90%, at least 95%, at least 98%, at least 99% or 100% identity to SEQ ID NO:3, wherein the nucleotide sequence encodes for succinic semialdehyde dehydrogenase (SSADH).

18. An adeno-associated virus (AAV) vector comprising the nucleic acid of claim 17.

19. An adeno-associated virus (AAV) particle comprising the AAV vector of claim 18 encapsidated in an AAV capsid.

20. The AAV particle of claim 19, wherein the AAV capsid comprises capsid proteins derived from AAV9 serotype.

21. The AAV particle of claim 19, wherein the AAV capsid comprises a capsid protein variant derived from AAV9.

22. The AAV particle of claim 21, wherein the capsid protein variant derived from AAV9 comprises PHP.B capsid or PHP.eB capsid.

23. The AAV particle of claim 19 for use in a method of treating succinic semialdehyde dehydrogenase deficiency (SSADHD) in a subject.

24. A composition comprising the AAV particle of claim 19.

25. A method of treating succinic semialdehyde dehydrogenase deficiency (SSADHD) in a subject, the method comprising administering to the subject an effective amount of the AAV particle of claim 19.


SEQUENCE LISTING (7 sequences)

SEQ ID NO: 1 - DNA, 1608 bp, Homo sapiens
  source 1..1608: mol_type = other DNA
atggctacatgtatttggcttcgaagttgtggagctcgacgacttggatctacatttccaggatgtcgacttcgacctcgagctggaggacttgttcctgcttctggacctgctcctggacctgctcaacttcgatgttatgctggacgacttgctggactttctgctgcacttcttcgaacagatagttttgttggaggacgatggcttcctgctgctgcaacatttcctgttcaagatcctgctagtggagctgctcttggaatggtagctgattgtggagttcgagaagctcgagctgctgttcgagctgcttatgaagcattctgccgatggagagaagtctccgccaaggagaggagttcattacttcggaagtggtacaatttaatgatacaaaataaggatgaccttgccagaataatcacagctgaaagtggaaagccactgaaggaggcacatggagaaattctctattccgcctttttcctagagtggttctctgaggaagcccgccgtgtttacggagacattatccacaccccggcaaaggacaggcgggccctggtcctcaagcagcccataggcgtggctgcagtcatcaccccgtggaatttccccagtgccatgatcacccggaaggtgggggccgccctggcagccggctgtactgtcgtggtgaagcctgccgaagacacgcccttctccgccctggccctggctgagcttgcaagccaggctgggattccttcaggtgtatacaatgttattccctgttctcgaaagaatgccaaggaagtaggggaggcaatttgtactgatcctctggtgtccaaaatttcctttactggttcaacaactacaggaaagatcctgttgcaccacgcagcaaactctgtgaaaagggtctctatggagctgggcggccttgctccatttatagtatttgacagtgccaacgtggaccaggctgtagcaggggccatggcatctaaatttaggaacactggacagacttgtgtttgctcaaaccaattcttggtgcaaaggggcatccatgatgcctttgtaaaagcattcgccgaggccatgaagaagaacctgcgcgtaggtaatggatttgaggaaggaactactcagggcccattaattaatgaaaaagcggtagaaaaggtggagaaacaggtgaatgatgccgtttctaaaggtgccaccgttgtgacaggtggaaaacgacaccaacttggaaaaaatttctttgagcctaccctgctgtgcaatgtcacccaggacatgctgtgcactcatgaagagactttcgggcctctggcaccagttatcaagttcgatacagaggaggaggctatagcaatcgctaacgcagctgatgttgggttagcaggttatttttactctcaagacccagcccagatctggagagtggcagagcagctggaagtgggcatggttggcgtcaacgaaggattaatttcctctgtggagtgcccttttggtggagtgaagcagtccggccttgggcgagaggggtccaagtatggcattgatgagtatctggaactcaagtatgtgtgttacgggggcttgtag

SEQ ID NO: 2 - DNA, 1850 bp, Homo sapiens
  source 1..1850: mol_type = genomic DNA
tggcttttctgggcctcagttgattgaggcctcaattgaatgatttccccaggccagtagaaatgagccctgtaatcccagcactttgggaggccaaggagggcggataacttgaggtcaggcattcaagaccagcctggccaacacggtgaaaacctgtctctactaaaaatacaaaaattagctgggcttggtggcacacacctgtagccccatctactcgggaggctgaggcaggagaatcgcttgaacccgggaggcggaagttgcagggagctgaaatcgcaccactgcactccaccctgggtgacagaatgagtctgtcggtctcacaagaacaaaacaaacaaaaaacaaaaaccaccaccaaaaacaaaacaaaacaaaaacaaaaactccctccgcaggcaggagatgtaaacacgccagctccccagccccacctgtgatacctactggcccacatccctattaggactccctggttctccccagttcctgcaataaaatacagagcaaaatagtgtgaaattgattaaactctactgttatcacttctgccatatgtgactggctcaaaactcaatggatctgactcagcaaaagacaaagtcaaaatttaaagttactgcctcggcattttgcaaatgtcctagaaaaaaattcaacgtcctagaaaaaggtgaaagctagcaagatactctcaacgctgtaggagttgagaaagggagatatgtaacctaaaatcaatgcatagggctgggcttcatttgcacccacaaattcccagcatacgcctgctatttttatttttatcctcattaggttttaagcttccctgctgctttcagagtcttaagcccaagtctaattttttaacggatgaagaatgctgtttaaaggtgctcaaataccttggttggatagtatgctcaatccttataccttggttgattcaattttgacaaatgcttccatttactagttggtgacccaactttaatcttactagtggcttgactagggtagctagtatgactttgaacgctatattgaatttaatgtggcaactgtaatgagcattttacgttatctcacaacaaaatggggtagatatggttctattttataagtgactggactaaggttaacctgtcggtcacacagcttgtcggcaacagacttgagtttggatcctagtctgtttatttccaaagcgctgttatttatcgggagcaaccctaggagaatgcctagacacacacccaaaaaagcagccaggcagcagaacgcggggtcacacttcgacccctcagagaacgatcgctcccaatcaatactacgtgctcaggagctacaactgaagaagcgtgatcacggccttggcatttagggcaggcaccagcgcatctatggaccgcgcacaaattccggggatgccgaatttgggggatgctaaggggagagagtgggtctctagcagcgattgggggctcaggagcagttagtgacaaatgagcacccgaaaagtgaaaaggtgacagcagtccgcaggtgcatctactggcgagccttctccatccccgaacccaaccctcccccgggagaaggtcgcgccaggagagaagccgcgcggcgcttagggcaaggtgcagagggcggcgcggcggtgcagcgagaaagacgcggagagagggcgctgctctgtggctctgcaaccttccgccagctcccacgctttccccgcgcgtccccggcgcctcctcgctcctcttgcttccccgcgacccctgcgttcccgtgcgcgcgcgcccgcttgcctgtttcctgtcgccgtcgttgcccgggcc

SEQ ID NO: 3 - DNA, 3458 bp, synthetic construct
  source 1..3458: mol_type = other DNA
tggcttttctgggcctcagttgattgaggcctcaattgaatgatttccccaggccagtagaaatgagccctgtaatcccagcactttgggaggccaaggagggcggataacttgaggtcaggcattcaagaccagcctggccaacacggtgaaaacctgtctctactaaaaatacaaaaattagctgggcttggtggcacacacctgtagccccatctactcgggaggctgaggcaggagaatcgcttgaacccgggaggcggaagttgcagggagctgaaatcgcaccactgcactccaccctgggtgacagaatgagtctgtcggtctcacaagaacaaaacaaacaaaaaacaaaaaccaccaccaaaaacaaaacaaaacaaaaacaaaaactccctccgcaggcaggagatgtaaacacgccagctccccagccccacctgtgatacctactggcccacatccctattaggactccctggttctccccagttcctgcaataaaatacagagcaaaatagtgtgaaattgattaaactctactgttatcacttctgccatatgtgactggctcaaaactcaatggatctgactcagcaaaagacaaagtcaaaatttaaagttactgcctcggcattttgcaaatgtcctagaaaaaaattcaacgtcctagaaaaaggtgaaagctagcaagatactctcaacgctgtaggagttgagaaagggagatatgtaacctaaaatcaatgcatagggctgggcttcatttgcacccacaaattcccagcatacgcctgctatttttatttttatcctcattaggttttaagcttccctgctgctttcagagtcttaagcccaagtctaattttttaacggatgaagaatgctgtttaaaggtgctcaaataccttggttggatagtatgctcaatccttataccttggttgattcaattttgacaaatgcttccatttactagttggtgacccaactttaatcttactagtggcttgactagggtagctagtatgactttgaacgctatattgaatttaatgtggcaactgtaatgagcattttacgttatctcacaacaaaatggggtagatatggttctattttataagtgactggactaaggttaacctgtcggtcacacagcttgtcggcaacagacttgagtttggatcctagtctgtttatttccaaagcgctgttatttatcgggagcaaccctaggagaatgcctagacacacacccaaaaaagcagccaggcagcagaacgcggggtcacacttcgacccctcagagaacgatcgctcccaatcaatactacgtgctcaggagctacaactgaagaagcgtgatcacggccttggcatttagggcaggcaccagcgcatctatggaccgcgcacaaattccggggatgccgaatttgggggatgctaaggggagagagtgggtctctagcagcgattgggggctcaggagcagttagtgacaaatgagcacccgaaaagtgaaaaggtgacagcagtccgcaggtgcatctactggcgagccttctccatccccgaacccaaccctcccccgggagaaggtcgcgccaggagagaagccgcgcggcgcttagggcaaggtgcagagggcggcgcggcggtgcagcgagaaagacgcggagagagggcgctgctctgtggctctgcaaccttccgccagctcccacgctttccccgcgcgtccccggcgcctcctcgctcctcttgcttccccgcgacccctgcgttcccgtgcgcgcgcgcccgcttgcctgtttcctgtcgccgtcgttgcccgggccatggctacatgtatttggcttcgaagttgtggagctcgacgacttggatctacatttccaggatgtcgacttcgacctcgagctggaggacttgttcctgcttctggacctgctcctggacctgctcaacttcgatgttatgctggacgacttgctggactttctgctgcacttcttcgaacagatagttttgttggaggacgatggcttcctgctgctgcaacatttcctgttcaagatcctgctagtggagctgctcttggaatggtagctgattgtggagttcgagaagctcgagctgctgttcgagctgcttatgaagcattctgccgatggagagaagtctccgccaaggagaggagttcattacttcggaagtggtacaatttaatgatacaaaataaggatgaccttgccagaataatcacagctgaaagtggaaagccactgaaggaggcacatggagaaattctctattccgcctttttcctagagtggttctctgaggaagcccgccgtgtttacggagacattatccacaccccggcaaaggacaggcgggccctggtcctcaagcagcccataggcgtggctgcagtcatcaccccgtggaatttccccagtgccatgatcacccggaaggtgggggccgccctggcagccggctgtactgtcgtggtgaagcctgccgaagacacgcccttctccgccctggccctggctgagcttgcaagccaggctgggattccttcaggtgtatacaatgttattccctgttctcgaaagaatgccaaggaagtaggggaggcaatttgtactgatcctctggtgtccaaaatttcctttactggttcaacaactacaggaaagatcctgttgcaccacgcagcaaactctgtgaaaagggtctctatggagctgggcggccttgctccatttatagtatttgacagtgccaacgtggaccaggctgtagcaggggccatggcatctaaatttaggaacactggacagacttgtgtttgctcaaaccaattcttggtgcaaaggggcatccatgatgcctttgtaaaagcattcgccgaggccatgaagaagaacctgcgcgtaggtaatggatttgaggaaggaactactcagggcccattaattaatgaaaaagcggtagaaaaggtggagaaacaggtgaatgatgccgtttctaaaggtgccaccgttgtgacaggtggaaaacgacaccaacttggaaaaaatttctttgagcctaccctgctgtgcaatgtcacccaggacatgctgtgcactcatgaagagactttcgggcctctggcaccagttatcaagttcgatacagaggaggaggctatagcaatcgctaacgcagctgatgttgggttagcaggttatttttactctcaagacccagcccagatctggagagtggcagagcagctggaagtgggcatggttggcgtcaacgaaggattaatttcctctgtggagtgcccttttggtggagtgaagcagtccggccttgggcgagaggggtccaagtatggcattgatgagtatctggaactcaagtatgtgtgttacgggggcttgtag

SEQ ID NO: 4 - DNA, 47 bp, Homo sapiens
  source 1..47: mol_type = other DNA
aaacgcgtctagtatgactttgaacgctatattgaatttaatgtggc

SEQ ID NO: 5 - DNA, 39 bp, Homo sapiens
  source 1..39: mol_type = other DNA
aagaattcaatctagacaacgacggcgacaggaaacagg

SEQ ID NO: 6 - DNA, 35 bp, Homo sapiens
  source 1..35: mol_type = other DNA
aatctagagccaccatggcgacctgcatttggctg

SEQ ID NO: 7 - DNA, 30 bp, Homo sapiens
  source 1..30: mol_type = other DNA
aagaattcctacaagcccccgtaacacaca